(19) United States
(12) Patent Application Publication
(10) Pub. No.: US 2021/0133635 A1
(43) Pub. Date: May 6, 2021

(54) MATERIAL DESCRIPTOR GENERATION METHOD, MATERIAL DESCRIPTOR GENERATION DEVICE, RECORDING MEDIUM STORING MATERIAL DESCRIPTOR GENERATION PROGRAM, PREDICTIVE MODEL CONSTRUCTION METHOD, PREDICTIVE MODEL CONSTRUCTION DEVICE, AND RECORDING MEDIUM STORING PREDICTIVE MODEL CONSTRUCTION PROGRAM

(71) Applicant: Panasonic Intellectual Property Management Co., Ltd., Osaka (JP)

(72) Inventors: REIKO HAGAWA, Osaka (JP); KOJI MORIKAWA, Tokyo (JP); HIROMASA TAMAKI, Osaka (JP)

(21) Appl. No.: 17/147,506

(22) Filed: Jan. 13, 2021

Related U.S. Application Data
(63) Continuation of application No. PCT/JP2019/028602, filed on Jul. 22, 2019.

(30) Foreign Application Priority Data
Aug. 8, 2018 (JP) 2018-149673
Mar. 29, 2019 (JP) 2019-066367

(51) Int. Cl. G06N 20/00; G06K 9/62

(52) U.S. Cl. CPC G06N 20/00; G06K 9/6232; G06K 9/6259

(57) ABSTRACT

A material descriptor generation method includes: acquiring a composition formula of a material; generating, from the composition formula, a formula expressing a base material and a dopant list including one or more formulas expressing one or more dopants used to dope the base material; computing descriptors needed to predict a predetermined property value of the material, the descriptors corresponding to the dopant list and the formula expressing the base material; and outputting a material descriptor consolidating the descriptors. The material descriptor is input into a predictive model that predicts the predetermined property value of the material.

### BACKGROUND

### 1. Technical Field

The present disclosure relates to a material descriptor generation method, a material descriptor generation device, and a recording medium storing a material descriptor generation program that generate descriptors to be input into a predictive model that predicts a predetermined property value of a material. The present disclosure also relates to a predictive model construction method, a predictive model construction device, and a recording medium storing a predictive model construction program that construct a predictive model that predicts a predetermined property value of a material.

### 2. Description of the Related Art

In the related art, it is possible to predict material properties with a simulation system such as first-principles calculation. In the simulation system, a property of a material is predicted by performing detailed physical calculation, but the calculation may take from several hours to several months in some cases. In contrast, in recent years attention has been focused on a method of predicting a property value of a material easily and quickly through machine learning or by constructing a logical model formula that accepts basic information about the material as input, and outputs a property value.

For example, there is a technology that accurately derives a property value of a material, namely the formation energy, by using descriptors computed from known parameters about the elements forming the material as input, as disclosed in A. Seko, H. Hayashi, K. Nakayama, A. Takahashi, and I. Tanaka, “Representation of compounds for machine-learning prediction of physical properties”, Physical Review B95, 144110, 2017. As another example, there is a technology that successfully predicts a property value of a material containing a dopant by devising a method of computing the descriptors computed from known parameters about the elements forming the material, as disclosed in A. Furmanchuk, J. E. Saal, J. W. Doak, G. B. Olson, A. Choudhary, and A. Agrawal, “Prediction of Seebeck Coefficient for Compounds without Restriction to Fixed Stoichiometry: A Machine Learning Approach”, Journal of Computational Chemistry 39(4), Feb. 5, 2018, pp. 191-202.

### SUMMARY

However, the technology according to Furmanchuk et al. needs further improvement.

One non-limiting and exemplary embodiment provides a technology that improves the performance for predicting a property value of a material.

In one general aspect, the techniques disclosed here feature a material descriptor generation method including acquiring a composition formula of a material, generating, from the composition formula, a formula expressing a base material and a dopant list including one or more formulas expressing one or more dopants used to dope the base material, computing descriptors needed to predict a predetermined property value of the material, the descriptors corresponding to the dopant list and the formula expressing the base material, and outputting a material descriptor consolidating the descriptors, in which the material descriptor is input into a predictive model that predicts the predetermined property value of the material.

It should be noted that general or specific embodiments may be implemented as an apparatus, a system, an integrated circuit, a computer program, a computer-readable recording medium, or any selective combination thereof. Computer-readable recording media include non-volatile recording media such as compact disc-read-only memory (CD-ROM), for example.

According to the present disclosure, the performance for predicting a property value of a material is improved by inputting a descriptor that clearly expresses a change in the type or quantity of dopant into a predictive model.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram for describing a procedure for predicting a property of a material;

FIG. 2 is a table illustrating an example of changes in a thermoelectric property (power factor) due to differences in a doping element and quantity of dopant used to dope a base material;

FIG. 3 is a diagram illustrating an example of a descriptor computed in Furmanchuk et al.;

FIG. 4 is a diagram illustrating a specific example of a descriptor computed according to the method in Furmanchuk et al.;

FIG. 5 is a diagram illustrating an example of a material descriptor in the present disclosure;

FIG. 6 is a diagram illustrating a specific example of a descriptor proposed by the present disclosure;

FIG. 7 is a diagram illustrating a configuration of a material property value prediction device in Embodiment 1;

FIG. 8 is a schematic diagram for explaining specific differences between a composition formula discrimination process according to Embodiment 1 and a composition formula discrimination process according to the related art;

FIG. 9 is a flowchart for explaining operations by the material property value prediction device in Embodiment 1;

FIG. 10 is a diagram illustrating an example of property value prediction or machine learning by a neural network using base material descriptors and dopant descriptors;

FIG. 11 is a flowchart for explaining the generation process in step S302 of FIG. 9 in Embodiment 1;

FIG. 12 is a diagram illustrating an example of a material descriptor including a descriptor computed from test environment information;

FIG. 13 is a diagram illustrating an example of a material descriptor including descriptors indicating coefficients of atomic symbols included in a formulas expressing dopants;

FIG. 14 is a diagram illustrating an example of a material descriptor including a descriptor that indicates a ratio of an atomic symbol included in a composition formula of a dopant with respect to the sum of the coefficients of all atomic symbols included in an input composition formula;

FIG. 15 is a diagram illustrating an example of a material descriptor including a coefficient of a host;

FIG. 16 is a diagram illustrating an example of a material descriptor in which zero or an average value is placed in a location where a descriptor calculated or determined from a formula expressing a dopant should be placed;

FIG. 17 is a diagram illustrating another example of a material descriptor in which zero or an average value is placed in a location where a descriptor calculated or determined from a formula expressing a dopant should be placed;

FIG. 18 is a diagram illustrating an example of property value prediction or machine learning by a neural network using base material descriptors, dopant descriptors, and test environment descriptors;

FIG. 19 is a diagram illustrating an example of multilevel machine learning by a neural network using base material descriptors and dopant descriptors;

FIG. 20 is a diagram illustrating a configuration of a material property value prediction device in Embodiment 2;

FIG. 21 is a flowchart for explaining the generation process in step S302 of FIG. 9 in Embodiment 2;

FIG. 22 is a diagram illustrating a configuration of a material property value prediction device in Embodiment 3;

FIG. 23 is a flowchart for explaining the generation process in step S302 of FIG. 9 in Embodiment 3;

FIG. 24 is a table illustrating the results of an experiment in Embodiment 3;

FIG. 25 is a diagram explaining the concept of a neural network device in Embodiment 4;

FIG. 26 is a diagram illustrating a configuration of a material property value prediction device in Embodiment 4;

FIG. 27 is a flowchart for explaining operations in a training mode by the material property value prediction device in Embodiment 4;

FIG. 28 is a flowchart for explaining the training process in step S1306 of FIG. 27 in Embodiment 4;

FIG. 29 is a flowchart for explaining operations in a prediction mode by the material property value prediction device in Embodiment 4; and

FIG. 30 is a diagram illustrating an example of a material descriptor in the present disclosure.

### DETAILED DESCRIPTION

### (Underlying Knowledge Forming Basis of the Present Disclosure)

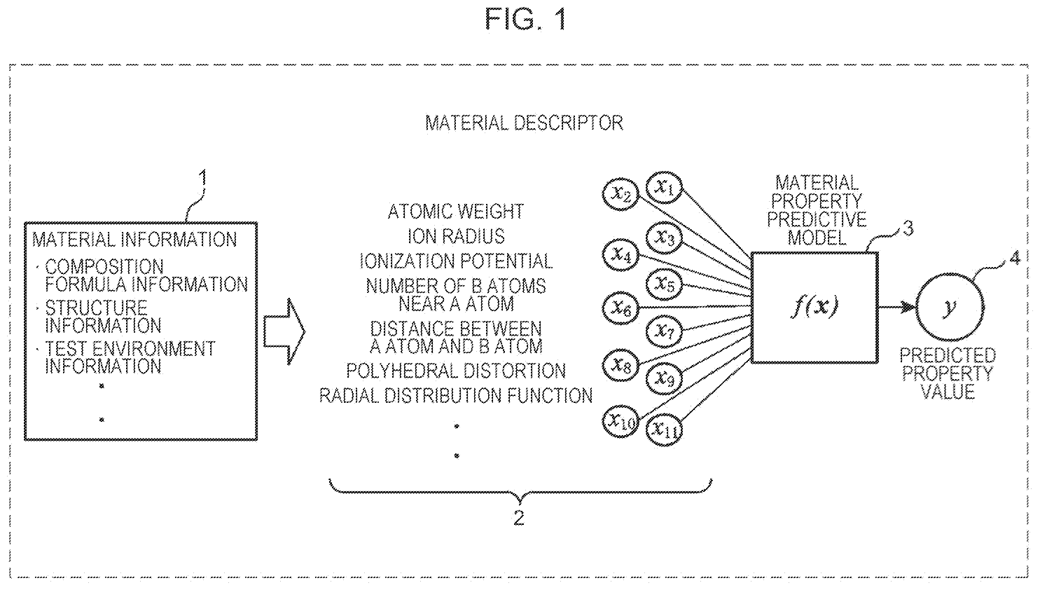

In recent years, attention has been focused on a method of predicting a property value of a material easily and quickly through machine learning or by constructing a logical model formula that accepts basic information about the material as input, and outputs a property value. A general procedure for predicting a property of a material through machine learning will be described using FIG. 1.

FIG. 1 is a diagram for describing a procedure for predicting a property of a material. First, a material descriptor 2 is derived from material information 1. The material information 1 includes composition formula information indicating a composition formula of the material, structure information indicating the structure of the material, test environment information indicating the environment in which the material is generated, and known parameters for each element, for example. Meanwhile, the material descriptor 2 expresses the information included in the material information 1 as numerical values, and is similar to the pixel values of an image. The material descriptor 2 is derived by combining known parameters of each element, such as atomic weights or ion radii, on the basis of the composition formula information, for example.

For example, in Seko et al. cited above, values such as a weighted average, a maximum value, or a minimum value of known parameters specific to each element are derived, and these values are used as the descriptor. Here, the known parameters specific to each element refer to a set of known numerical values for each element that are acquirable without performing physical calculations, such as the atomic volume, covalent radius, or density. Also, the weighted average of the parameters is computed on the basis of the number of atoms forming the material. For example, the weighted average of the atomic radii of “CaMnO₃” is obtained by weighting the atomic radius of 197 for Ca, the atomic radius of 127 for Mn, and the atomic radius of 60 for O according to the ratio “Ca:Mn:O=1:1:3”. In other words, the weighted average of the atomic radii of “CaMnO₃” is (197+127+60*3)/5=100.8. The material descriptor 2 is input into a material property predictive model 3. The material property predictive model 3 predicts a property of the material and outputs a predicted property value 4.

Generally, in material property prediction, a property value of a substance without any impurities (hereinafter referred to as a base material) is predicted. However, in the field of semiconductor materials, base materials are often doped with a dopant, thereby the property values of the material being changed greatly.

Inventors have recognized the need to propose a method of generating a descriptor capable of clearly expressing even small changes in the type or quantity of dopant. Hereinafter, this line of thinking will be described.

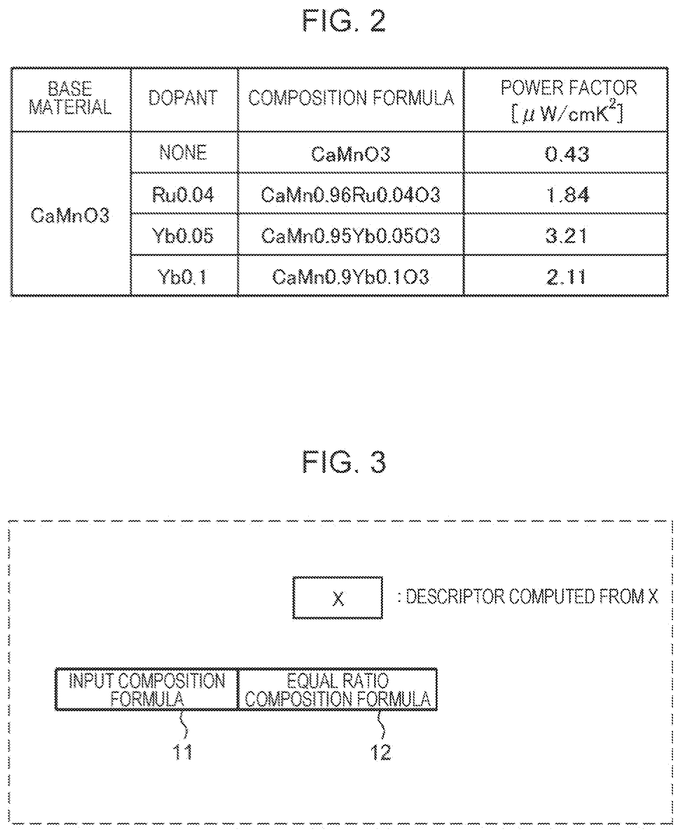

FIG. 2 is a table illustrating an example of changes in a thermoelectric property (power factor) due to differences in the type and quantity of dopant element used to dope the base material CaMnO₃. Note that in FIG. 2, the power factor of each material is measured under temperature conditions of 1000 K. As FIG. 2 demonstrates, the value of the power factor is the small value of 0.43 in the case where nothing is added as a dopant to the base material CaMnO₃, but by adding Ru or Yb as a dopant to the base material, the value of the power factor is improved. FIG. 2 also demonstrates that adding Yb_{0.05} as the dopant raises the value of the power factor by approximately 1.7 times compared to the case of adding Ru_{0.04}. Furthermore, even if the same Yb is used, adding Yb_{0.1} lowers the value of the power factor to approximately ⅔rds compared to the case of adding Yb_{0.05}. In this way, the property value of a material may change greatly if the type or quantity of dopant element is even slightly different. For this reason, when the type or quantity of dopant element changes, it is necessary to generate a descriptor capable of clearly expressing the difference in the type or quantity of dopant element.

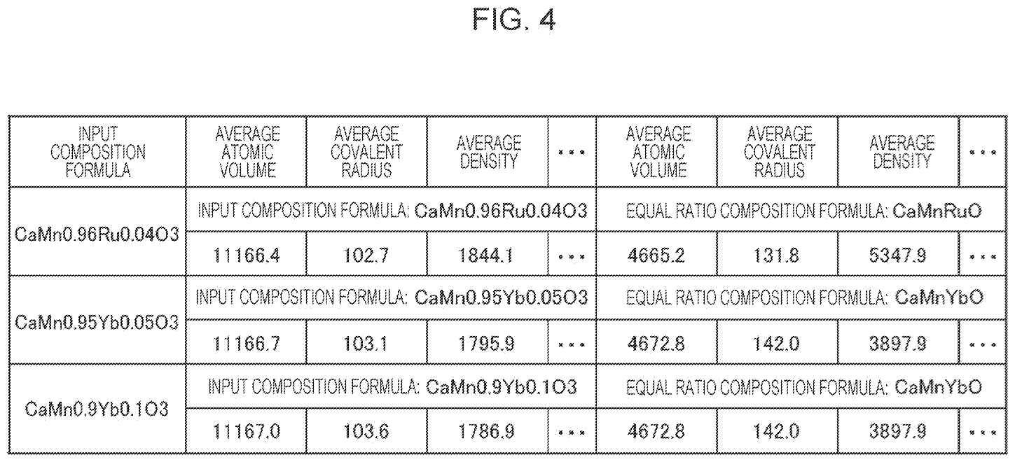

Because a descriptor derived using the technology in Furmanchuk et al. simply averages the element information irrespectively of the base material and the dopant, the difference cannot be expressed clearly if there is a small change in the type or quantity of dopant element. If the type or quantity of the dopant element is slightly different, the dopant may exert a large influence on the property values of the material. For this reason, data that clearly expresses changes in the type or quantity of dopant element cannot be used to create a predictive model by training a neural network device, for example, and the performance of the neural network device in predicting a property value of the material is reduced. For this reason, the method of generating a descriptor capable of clearly expressing even small changes in the type or quantity of dopant element needs further improvement. Hereinafter, an examination of Furmanchuk et al. will be described in detail. First, FIGS. 3 and 4 will be used to describe the method of deriving a descriptor from a composition formula containing dopant information in Furmanchuk et al. In Furmanchuk et al., an equal ratio composition formula is derived from an input composition formula, a weighted average or the standard deviation of information about each element is calculated for both the input composition formula and the equal ratio composition formula, and the calculated values are used as a descriptor.

FIG. 3 is a diagram illustrating an example of a descriptor computed in Furmanchuk et al. In FIG. 3, a descriptor 11 computed from an input composition formula and a descriptor 12 computed from an equal ratio composition formula are concatenated and converted into a single sequence. Here, in the case where the composition formula is “CaMn_{0.96}Ru_{0.04}O₃” for example, the equal ratio composition formula refers to the composition formula “CaMnRuO” in which the coefficients of all elements are set to 1, irrespectively of the classification of base material and dopant.

FIG. 4 is a diagram illustrating a specific example of a descriptor computed according to the method in Furmanchuk et al. As illustrated in FIG. 2, in a semiconductor material, not only the base material but also the dopant element and the dopant quantity influence the properties. The descriptor of the related art generated from the equal ratio composition formula is capable of expressing changes caused by the dopant element. In the example of FIG. 4 as well, each descriptor changes according to the dopant element, and in the case of a descriptor with a large change, a change of several tens of percent is demonstrated. However, it is difficult for the descriptor of the related art generated from the input composition formula to clearly express changes in the dopant quantity. In the example of FIG. 4, each descriptor changes only slightly with respect to a change in the dopant element or the dopant quantity, and even in the case of a descriptor with a large change, the change is only a few percent of the total quantity. The descriptor of the related art generated from the input composition formula is incapable of clearly expressing slight changes in the quantity of the dopant element that influences the property values.

A material descriptor generation method according to one aspect of the present disclosure includes: acquiring a composition formula of a material; generating, from the composition formula, a formula expressing a base material and a dopant list including one or more formulas expressing one or more dopants used to dope the base material; computing descriptors needed to predict a predetermined property value of the material, the descriptors corresponding to the dopant list and the formula expressing the base material; and outputting a material descriptor consolidating the descriptors, wherein the material descriptor is input into a predictive model that predicts the predetermined property value of the material.

According to this configuration, because a formula expressing a base material and a dopant list including one or more formulas expressing one or more dopants used to dope the base material are generated from a composition formula of a material, and because descriptors needed to predict a predetermined property value of the material and corresponding to the formula expressing the base material and the dopant list are computed, descriptors that clearly express changes in the one or more types or the one or more quantities of the one or more dopants can be generated, even for a material in which the one or more types or the one or more quantities of the one or more dopants changes slightly. Also, by inputting a material descriptor consolidating the descriptors that clearly express changes in the type or quantity of dopant(s) into a predictive model, the performance for predicting a property value of the material can be improved.

The above material descriptor generation method may also be configured such that the generating of the formula expressing the base material and the dopant list includes acquiring a base material list including formulas expressing base materials, computing a composition difference value between each of the formulas expressing the base materials and the composition formula, acquiring a minimum composition difference value that is the smallest composition difference value among the computed composition difference values and a first formula expressing a first base material used to compute the minimum composition difference value, the formulas expressing the base materials including the first formula expressing the first base material, determining whether or not the minimum composition difference value is a threshold value or less, in a case of determining that the minimum composition difference value is greater than the threshold value, applying a rejection label to the composition formula, in a case of determining that the minimum composition difference value is the threshold value or less, acquiring a differential composition formula expressing a formula of a difference between the first formula and the composition formula, and generating a second formula in accordance with the differential composition formula. The one or more formulas expressing the one or more dopants include the second formula.

According to this configuration, by computing a composition difference value between the composition formula and each of the formulas expressing the base materials included in the base material list, composition difference values are computed. Additionally, it is determined whether or not the minimum composition difference value, that is, the smallest composition difference value among the computed composition difference values, is a threshold value or less. At this time, in the case where the minimum composition difference value is greater than the threshold value, the quantity of element included in the formula expressing the dopant that is the difference between the formula expressing the base material and the composition formula is more than the quantity of element included in the formula expressing the base material, and therefore the formula expressing the base material and the formula expressing the dopant cannot be discriminated appropriately, and the composition formula can be determined to be inappropriate. Consequently, by applying a rejection label to the composition formula in the case of determining that the minimum composition difference value is greater than the threshold value, it is possible to keep an inappropriate composition formula from being adopted. Also, in the case where the minimum composition difference value is the threshold value or less, a formula expressing the dopant can be specified from a differential composition formula expressing the differential composition between the formula expressing the base material and the composition formula. Also, in the case of determining that the minimum composition difference value is the threshold value or less, a second formula is generated on the basis of the differential composition formula expressing the differential composition between the formula expressing the base material and the composition formula, the first formula expressing the base material used when computing the minimum composition difference value and the generated dopant list are output, and in addition, because the one or more formulas expressing the one or more dopants include the second formula, the first formula expressing the base material and the dopant list can be discriminated appropriately.

The above material descriptor generation method may also be configured such that the generating of the formula expressing the base material and the dopant list includes selecting an atomic symbol and a coefficient of the atomic symbol from the composition formula, determining whether or not the coefficient is greater than a threshold value, in a case of determining that the coefficient is the threshold value or less, adding the atomic symbol to the dopant list, in a case of determining that the coefficient is greater than the threshold value, adding a combined formula that combines the atomic symbol with a new coefficient generated by rounding up a fractional part of the coefficient to a base material element list, adding each atomic symbol to the dopant list or to the base material element list for all atomic symbols included in the composition formula, thereby causing the base material element list to include combined formulas, each of which is the combined formula that combines the atomic symbol with the new coefficient generated by rounding up the fractional part of the coefficient, deriving a formula expressing a base material consolidating the combined formulas included in the base material element list, and outputting the formula expressing the base material and the dopant list.

According to this configuration, one atomic symbol and its coefficient are selected from the formula expressing the composition formula, and it is determined whether or not the selected coefficient is greater than a threshold value. In the case where the coefficient is the threshold value or less, the selected atomic symbol is added to the dopant list, and therefore the dopant list can be generated. In the case where the coefficient is greater than the threshold value, it is determined that the selected atomic symbol is included in the formula expressing the base material. In the case of determining that the coefficient is greater than the threshold value, a combined formula with a new coefficient generated by rounding up the fractional part of the coefficient is added to the base material element list. All atomic symbols included in the composition formula are added to the dopant list or added to the base material element list, and with this arrangement, because the base material element list includes the combined formulas, and because a formula expressing the base material is derived by consolidating the combined formulas included in the base material element list, the formula expressing the base material can be specified appropriately.

The above material descriptor generation method may also be configured such that the generating of the formula expressing the base material and the dopant list includes acquiring a base material list including formulas expressing base materials, determining whether or not a sum of coefficients of atomic symbols in the composition formula is an integer, in a case of determining that the sum is an integer, selecting an atomic symbol and a coefficient of the atomic symbol from the composition formula, determining whether or not the coefficient is greater than a threshold value, in a case of determining that the coefficient is the threshold value or less, adding the atomic symbol to the dopant list, in a case of determining that the coefficient is greater than the threshold value, adding a combined formula that combines the atomic symbol with a new coefficient generated by rounding up a fractional part of the coefficient to a base material element list, adding each atomic symbol to the dopant list or to the base material element list for all atomic symbols included in the composition formula, thereby causing the base material element list to include combined formulas, each of which is the combined formula that combines the atomic symbol with the new coefficient generated by rounding up the fractional part of the coefficient, deriving a formula expressing a base material consolidating the combined formulas included in the base material element list, determining whether or not the formula expressing the base material that is derived exists in the base material list, in a case of determining that the formula expressing the base material exists in the base material list, outputting the formula expressing the base material and the dopant list, and in a case of determining that the sum is not an integer, or in a case of determining that the formula expressing the base material does not exist in the base material list, applying a rejection label to the composition formula.

According to this configuration, if the sum of the coefficients of the atomic symbols in the composition formula is an integer, one atomic symbol and its coefficient are selected from the composition formula, and it is determined whether or not the selected coefficient is greater than a threshold value. In the case where the coefficient is the threshold value or less, the selected atomic symbol is added to the dopant list, and therefore the dopant list can be generated. In the case where the coefficient is greater than the threshold value, it is determined that the selected atomic symbol is an element forming the base material. In the case of determining that the coefficient is greater than the threshold value, a combined formula with a new coefficient generated by rounding up the fractional part of the coefficient is added to the base material element list. All atomic symbols included in the composition formula are added to the dopant list or added to the base material element list, and with this arrangement, because the base material element list includes the combined formulas, and because a base material is derived by consolidating the elements included in the base material element list, the formula expressing the base material can be specified appropriately. Furthermore, because it is determined whether or not a formula expressing the derived base material exists in the base material list, a formula expressing the materials that actually exist as the base material can be output, and the accuracy of discriminating between the formula expressing the base material and the dopant list can be improved.

The above material descriptor generation method may also be configured to further include acquiring environment information indicating an environment where the material is generated, wherein the computing of the descriptors includes computing a descriptor corresponding to the environment information.

According to this configuration, because environment information expressing the environment in which the material is generated is acquired, and because a descriptor corresponding to the environment information is computed, the environment in which the material is generated can be taken into consideration to predict the predetermined property value of the material.

The above material descriptor generation method may also be configured to further include acquiring structure information indicating a structure of the material, wherein the computing of the descriptors includes computing a descriptor corresponding to the structure information.

According to this configuration, structure information expressing the structure of the material is acquired and a descriptor corresponding to the structure information is computed, and therefore the structure of the material can be taken into consideration to predict the predetermined property value of the material.

The above material descriptor generation method may also be configured such that the computing of the descriptors generates a coefficient of a formula expressing a dopant included in the one or more formulas expressing the one or more dopants as a descriptor.

According to this configuration, the coefficients of a formula expressing a dopant included in one or more formulas expressing one or more dopants can be taken into consideration to predict the predetermined property value of the material.

The above material descriptor generation method may also be configured such that the computing of the descriptors generates, as a descriptor, a numerical value obtained by dividing each of one or more coefficients of the one or more formulas expressing the one or more dopants included in the dopant list by a sum of all coefficients included in the composition formula.

According to this configuration, a numerical value obtained by dividing each of one or more coefficients of one or more formulas expressing one or more dopants included in the dopant list by the sum of all coefficients included in the composition formula can be taken into consideration to predict the predetermined property value of the material.

The above material descriptor generation method may also be configured such that in a case where a second coefficient is decreased due to increasing a first coefficient, the computing of the descriptors generates a coefficient indicating an amount of the decrease as a descriptor, and the one or more formulas expressing the one or more dopants includes a first atomic symbol having the first coefficient and a second atomic symbol having the second coefficient.

According to this configuration, the one or more formulas expressing one or more dopants include a first atomic symbol having a first coefficient and a second atomic symbol having a second coefficient, and in the case where the second coefficient is decreased by increasing the first coefficient, a coefficient expressing the decreased amount can be taken into consideration to predict the predetermined property value of the material.

A material descriptor generation device according to another aspect of the present disclosure includes: an acquirer that acquires a composition formula of a material; a discriminator that discriminates, from the composition formula, a formula expressing a base material and a dopant list including one or more formulas expressing one or more dopants used to dope the base material; a calculator that computes descriptors needed to predict a predetermined property value of the material, the descriptors corresponding to the dopant list and the formula expressing the base material; and an outputted that outputs a material descriptor consolidating the descriptors, wherein the material descriptor is input into a predictive model that predicts the predetermined property value of the material.

According to this configuration, because a formula expressing a base material and a dopant list including one or more formulas expressing one or more dopants used to dope the base material are generated from a composition formula of a material, and because descriptors needed to predict a predetermined property value of the material and corresponding to the formula expressing the base material and the dopant list are computed, descriptors that clearly express changes in the one or more types or the one of more quantities of the one or more dopants can be generated, even for a material in which the one or more types or the one of more quantities of the one or more dopants changes slightly. Also, by inputting a material descriptor consolidating the descriptors that clearly express changes in the type or quantity of dopant(s) into a predictive model, the performance for predicting a property value of the material can be improved.

A non-transitory computer-readable recording medium storing a material descriptor generation program according to another aspect of the present disclosure causes a computer to execute a process including: acquiring a composition formula of a material; generating, from the composition formula, a formula expressing a base material and a dopant list including one or more formulas expressing one or more dopants used to dope the base material; computing descriptors needed to predict a predetermined property value of the material, the descriptors corresponding to the dopant list and the formula expressing the base material; and outputting a material descriptor consolidating the descriptors, wherein the material descriptor is input into a predictive model that predicts the predetermined property value of the material.

According to this configuration, because a formula expressing a base material and a dopant list including one or more formulas expressing one or more dopants used to dope the base material are generated from a composition formula of a material, and because descriptors needed to predict a predetermined property value of the material and corresponding to the formula expressing the base material and the dopant list are computed, descriptors that clearly express changes in the one or more types or the one of more quantities of the one or more dopants can be generated, even for a material in which the one or more types or the one of more quantities of the one or more dopants changes slightly. Also, by inputting a material descriptor consolidating the descriptors that clearly express changes in the type or quantity of dopant(s) into a predictive model, the performance for predicting a property value of the material can be improved.

A predictive model construction method according to another aspect of the present disclosure is a predictive model construction method in a predictive model construction device that constructs a predictive model predicting a predetermined property value of a material, the method including: generating a descriptor indicating a predetermined feature of the material; and training the predictive model by using the descriptor as an input value.

According to this configuration, because a descriptor that clearly expresses a change in the type or quantity of dopant is generated even for a material for which the type or quantity of dopant changes slightly, and because a predictive model is trained by using the generated descriptor as an input value, the performance for predicting a property value of the material using the predictive model can be improved.

The above predictive model construction method may also be configured such that the generating of the descriptor includes acquiring a composition formula of the material, generating, from the composition formula, a formula expressing a base material and a dopant list including one or more formulas expressing one or more dopants used to dope the base material, computing descriptors needed to predict the predetermined property value, the descriptors corresponding to the dopant list and the formula expressing the base material, and outputting a material descriptor consolidating the descriptors.

According to this configuration, because a formula expressing a base material and a dopant list including one or more formulas expressing one or more dopants used to dope the base material are generated from the composition formula of a material, and because descriptors needed to predict a predetermined property value and corresponding to the formula expressing the base material and the dopant list are computed, a descriptor that clearly expresses a change in the type or quantity of dopant can be generated, even for a material in which the type or quantity of dopant changes slightly.

A predictive model construction device according to another aspect of the present disclosure constructs a predictive model predicting a predetermined property value of a predetermined material, and includes: a generator that generates a descriptor indicating a feature of the predetermined material; and a trainer that trains the predictive model by using the descriptor as an input value.

According to this configuration, because a descriptor that clearly expresses a change in the type or quantity of dopant is generated even for a material for which the type or quantity of dopant changes slightly, and because a predictive model is trained by using the generated descriptor as an input value, the performance for predicting a property value of the material using the predictive model can be improved.

A non-transitory computer-readable recording medium storing a predictive model construction program according to another aspect of the present disclosure causes a computer to execute a process of constructing a predictive model predicting a predetermined property value of a predetermined material, the process including: generating a descriptor indicating a feature of the predetermined material; and training the predictive model by using the descriptor as an input value.

According to this configuration, because a descriptor that clearly expresses a change in the type or quantity of dopant is generated even for a material for which the type or quantity of dopant changes slightly, and because a predictive model is trained by using the generated descriptor as an input value, the performance for predicting a property value of the material using the predictive model can be improved.

Hereinafter, embodiments of the present disclosure will be described with reference to the attached drawings. Note that the following embodiments are merely specific examples of the present disclosure, and do not limit the technical scope of the present disclosure.

### Embodiment 1

First, an overview of the descriptor proposed by the present disclosure will be described.

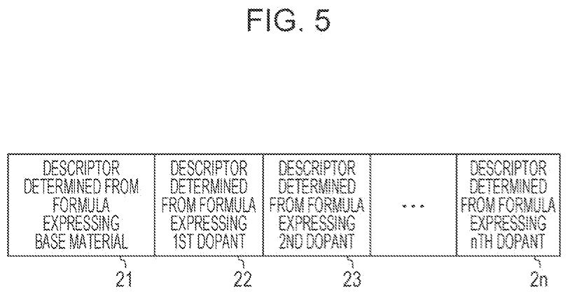

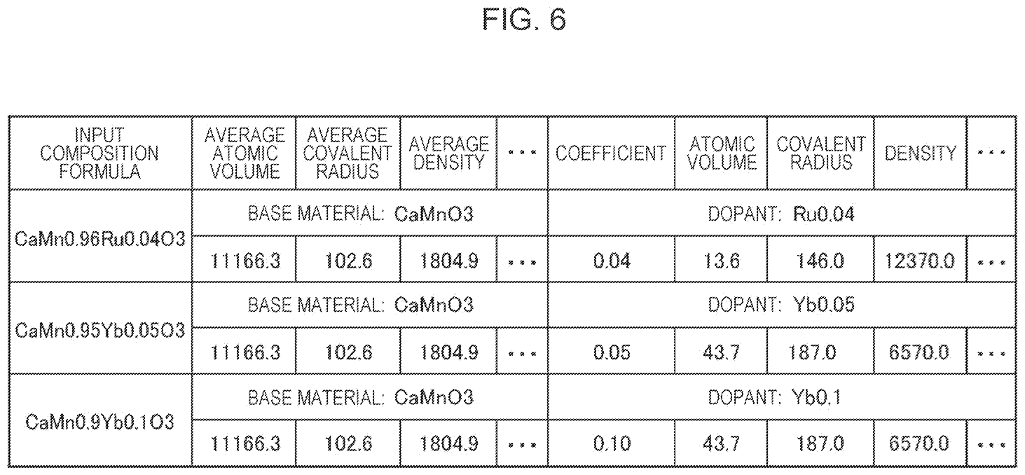

The present disclosure proposes a method of discriminating between a formula expressing a base material and a formula expressing a dopant from a composition formula of a material containing a dopant, and computing a descriptor from each of the discriminated formula expressing the base material and the discriminated formula expressing the dopant. An overview of the format of the descriptor proposed by the present disclosure will be described using FIGS. 5 and 6. Note that “computing a descriptor” may also be restated as “determining a descriptor”.

FIG. 5 is a diagram illustrating an example of a material descriptor in the present disclosure. The material descriptor includes descriptors, namely a descriptor 21 and descriptors 22 to 2n. As illustrated in FIG. 5, the descriptor 21 computed from a formula expressing a base material and each of the descriptors 22 to 2n computed or determined from formulas respectively expressing 1st to nth dopants are concatenated and converted into a single sequence.

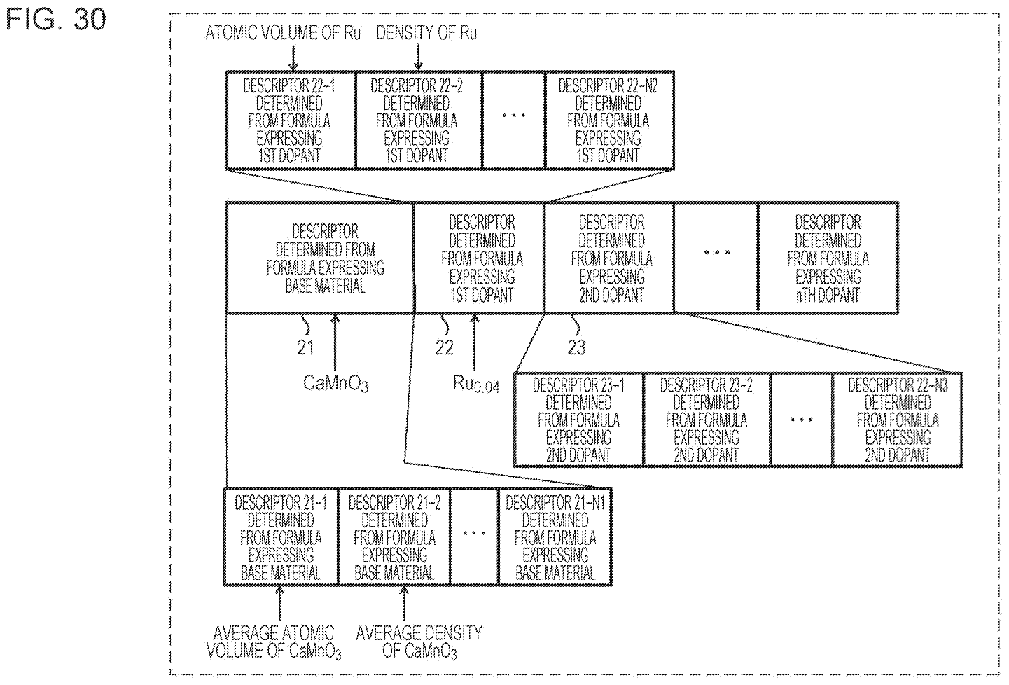

FIG. 30 is a diagram illustrating an example of a material descriptor in the present disclosure. In FIG. 30, the descriptor 21 computed from a formula expressing a base material may also be one or more descriptors 21-1, 21-2, and so on computed from the formula expressing the same base material. As illustrated in FIG. 30, each of the descriptors 22 to 2n computed from formulas expressing the 1st to nth dopants, respectively, may be one or more descriptors computed from the formula expressing the same dopant.

Note that in general, the base material refers to a material with zero chemical potential shift, but in Embodiment 1, for simplicity, a formula expressing a material having all-integer coefficients for the atomic symbols included in an input composition formula is defined as the formula expressing the base material.

In the case where an atomic symbol included in a composition formula has a coefficient of 1, “1” is generally not indicated, and in cases where an atomic symbol has no coefficient in the present specification, claims, drawings, and abstract, the coefficient may be assumed to be “1”. For example, “CaMnO₃” may be considered to be “Ca₁Mn₁O₃”.

FIG. 6 is a diagram illustrating a specific example of the descriptor proposed by the present disclosure.

An example of one or more descriptors computed from a formula expressing a base material CaMnO₃ is “11166.3”, “102.6”, and/or “1804.9”. Here, “11166.3” is the average atomic volume computed from the formula expressing the base material CaMnO₃, “102.6” is the average covalent radius computed from the formula expressing the base material CaMnO₃, and “1804.9” is the average density computed from the formula expressing the base material CaMnO₃.

An example of one or more descriptors computed or determined from a formula expressing a dopant Ru_{0.04} is “0.04”, “13.6”, “146.0”, and/or “12370.0”. Here, “0.04” is the coefficient of the dopant Ru_{0.04}, “13.6” is the atomic volume computed or determined from the formula expressing the dopant Ru_{0.04}, “146.0” is the covalent radius computed or determined from the formula expressing the dopant Ru_{0.04}, and “12370.0” is the density computed or determined from the formula expressing the dopant Ru_{0.04}.

As illustrated in FIG. 2, in the field of semiconductor materials, not only the base material but also the type and quantity of the dopant element influence the properties of the material. In Embodiment 1 of the present disclosure, the material descriptor includes a descriptor indicating information about the element of a dopant derived from a formula expressing the dopant, and a descriptor indicating information about the quantity of element in the dopant derived from the formula expressing the dopant.

The difference between elements of the dopants is clearly expressed by having the material descriptor include a descriptor using a known parameter specific to each element. As illustrated in FIG. 6, the known parameter specific to each element is the atomic volume, the covalent radius, or the density, for example. Also, the difference between quantities of the dopants is clearly expressed by having the material descriptor include a descriptor indicating the dopant coefficient. As illustrated in FIG. 6, in the case where the formula expressing the dopant is Ru_{0.04}, the dopant coefficient is 0.04.

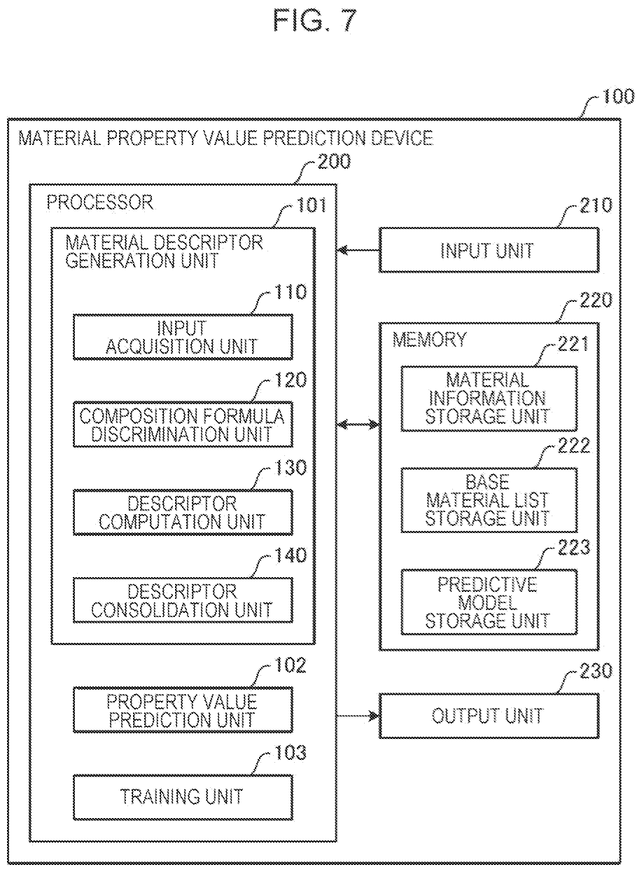

FIG. 7 is a diagram illustrating a configuration of a material property value prediction device in Embodiment 1. A material property value prediction device 100 in Embodiment 1 is a personal computer, for example, and includes a processor 200, an input unit 210, memory 220, and an output unit 230. The processor 200 includes a material descriptor generation unit 101, a property value prediction unit 102, and a training unit 103. Additionally, the material descriptor generation unit 101 includes an input acquisition unit 110, a composition formula discrimination unit 120, a descriptor computation unit 130, and a descriptor consolidation unit 140. The memory 220 includes a material information storage unit 221, a base material list storage unit 222, and a predictive model storage unit 223. The material property value prediction device 100 constructs a predictive model that predicts a predetermined property value of a material.

The material descriptor generation unit 101 generates a material descriptor to be input into the predictive model that predicts a predetermined property value of a material.

The input unit 210 includes a keyboard and mouse or a touch panel, for example, and receives various information input by a user. The input unit 210 receives the input of a composition formula by a user about which a predetermined property value is desired to be predicted. The composition formula received by the input unit 210 may also be referred to as the input composition formula. The composition formula input by the user may also be referred to as the input composition formula.

The material information storage unit 221 stores material information related to one or more materials. The material information includes composition formula information indicating one of more composition formulas corresponding to one or more materials, structure information indicating one or more structures of the one or more materials, and test environment information regarding the one or more materials. The test environment information regarding the one or more materials includes one or more environments where the one or more materials are generated, information about one or more temperatures when the properties of the one or more materials are measured, and/or one or more specific methods of generating the one or more materials. During training, material information that includes composition formula information, structure information, and test environment information for materials is used, and during prediction, material information that includes structure information and test environment information corresponding to composition formula information indicating the composition formula of the material input by the user is used.

The material information may include one or more known parameters of each element. Examples of the one or more known parameters of each element includes an atomic volume value, a covalent radius value, and a density value. The material information may include one or more known parameters for elements. Examples of the one or more known parameters for the elements includes an average atomic volume value, an average covalent radius value, and an average density value.

The base material list storage unit 222 stores a base material list describing formulas expressing base materials in advance. Note that in Embodiment 1, the base material list is stored in the base material list storage unit 222, but the present disclosure is not particularly limited thereto, and the base material list may also be received by a communication unit not illustrated from an external device over a network. The base material list may include formulas recorded in a predetermined database. The predetermined database is the Inorganic Crystal Structure Database (ICSD) described in A. Belsky, M. Hellenbrandt, V. L. Karen, and P. Luksch, “New developments in the Inorganic Crystal Structure Database (ICSD): accessibility in support of materials research and design”, 2002, Acta Cryst. B58, 364-369, for example. The base material list may also be generated in advance using the method illustrated in Embodiment 2.

The predictive model storage unit 223 stores a predictive model that predicts a predetermined property value of a material. The predictive model is for example a neural network that treats the material descriptor as input information and the predetermined property value as output information.

The input acquisition unit 110 receives the input composition formula from the input unit 210.

The composition formula discrimination unit 120 discriminates between a formula expressing a base material and one or more formulas expressing one or more dopants used to dope the base material from the input composition formula received from the input acquisition unit 110, and generates a dopant list that includes the one or more formulas expressing the one or more dopants.

The composition formula discrimination unit 120 acquires the base material list indicating formulas expressing base materials from the base material list storage unit 222. The composition formula discrimination unit 120 computes a composition difference value between each of the formulas expressing the base materials in the base material list and the input composition formula. Details about the composition difference value will be described later. The composition formula discrimination unit 120 acquires a minimum composition difference value from among the computed composition difference values, and the formula expressing the base material used to compute the minimum composition difference value. The composition formula discrimination unit 120 determines whether or not the minimum composition difference value is a threshold value or less. In the case of determining that the minimum composition difference value is greater than the threshold value, the composition formula discrimination unit 120 applies a rejection label to the composition formula and notifies the descriptor computation unit 130. In the case of determining that the minimum composition difference value is the threshold value or less, the composition formula discrimination unit 120 acquires a differential composition formula between the formula expressing the base material and the composition formula. From the differential composition formula, the composition formula discrimination unit 120 generates a dopant list including the one or more formulas of one or more dopants. The composition formula discrimination unit 120 outputs information including the formula expressing the base material and the dopant list.

In the case of being notified by the composition formula discrimination unit 120 of the rejection label being applied to the input composition formula, the descriptor computation unit 130 concludes that the formula expressing the base material and the dopant list have not been generated.

In the case where the formula expressing the base material and the dopant list have been generated, the descriptor computation unit 130 computes descriptors needed to predict the predetermined property value, the descriptors corresponding to the dopant list and the formula expressing the base material.

The descriptor consolidation unit 140 generates a material descriptor consolidating the descriptors computed by the descriptor computation unit 130 into a single sequence.

The property value prediction unit 102 uses the predictive model stored in the predictive model storage unit 223 to predict the predetermined property value on the basis of the material descriptor. The property value prediction unit 102 inputs the material descriptor into the predictive model read out from the predictive model storage unit 223, and obtains the predetermined property value output from the predictive model. The predetermined property value may be a value indicating the power factor or a value indicating the electrical resistivity of the material. Examples of a property item whose value is predicted include the power factor and the electrical resistivity.

The training unit 103 trains the predictive model using the material descriptor generated by the material descriptor generation unit 101 as an input value. The training unit 103 uses the material descriptor output from the descriptor consolidation unit 140 to perform machine learning on the predictive model stored in the predictive model storage unit 223. Examples of the machine learning include supervised learning in which labeled teaching data (that is, data having output information associated with input information) is used to learn the relationship between the input and the output, unsupervised learning in which a structure of the data is constructed from unlabeled data, semi-supervised learning in which both labeled and unlabeled data are handled, and reinforcement learning in which feedback (a reward) with respect to an action selected from a result of observing a state is obtained or consecutive actions that maximize the reward are learned. Additionally, specific methods of machine learning include a neural network (including deep learning using a multilayer neural network), genetic programming, a decision tree, a Bayesian network, or a support vector machine (SVM). In the machine learning according to the present disclosure, it is sufficient to use any of the specific examples mentioned above.

The material property value prediction device 100 in Embodiment 1 is capable of switching between a prediction mode that predicts a predetermined property value of a material and a training mode that trains the predictive model. In the prediction mode, the input acquisition unit 110 acquires an input composition formula input by the input unit 210. Meanwhile, in the training mode, machine learning is performed on the predictive model by causing the input acquisition unit 110 to acquire input composition formulas stored in advance in the material information storage unit 221 and by causing the training unit 103 to input each of the material descriptors computed from each of the input composition formulas into the predictive model.

The output unit 230 outputs the predetermined property value predicted by the property value prediction unit 102. Note that the output unit 230 may be a display device, and may display the property value predicted by the property value prediction unit 102. The output unit 230 may also be a printer, and may print the property value predicted by the property value prediction unit 102. Furthermore, the output unit 230 may also be an output terminal, and may output the property value predicted by the property value prediction unit 102 to an external destination.

Note that the material property value prediction device 100 may also be a server. In this case, the material property value prediction device 100 does not include the input unit 210 and the output unit 230 but further includes a communication unit, and is communicably connected to a terminal device. The terminal device includes the input unit 210 and the output unit 230, receives the input of a composition formula, and transmits the received composition formula to the material property value prediction device 100 as the input composition formula. The material property value prediction device 100 receives the input composition formula from the terminal device, predicts a predetermined property value on the basis of the received input composition formula, and transmits the predicted predetermined property value to the terminal device. The terminal device receives the predicted predetermined property value from the material property value prediction device 100.

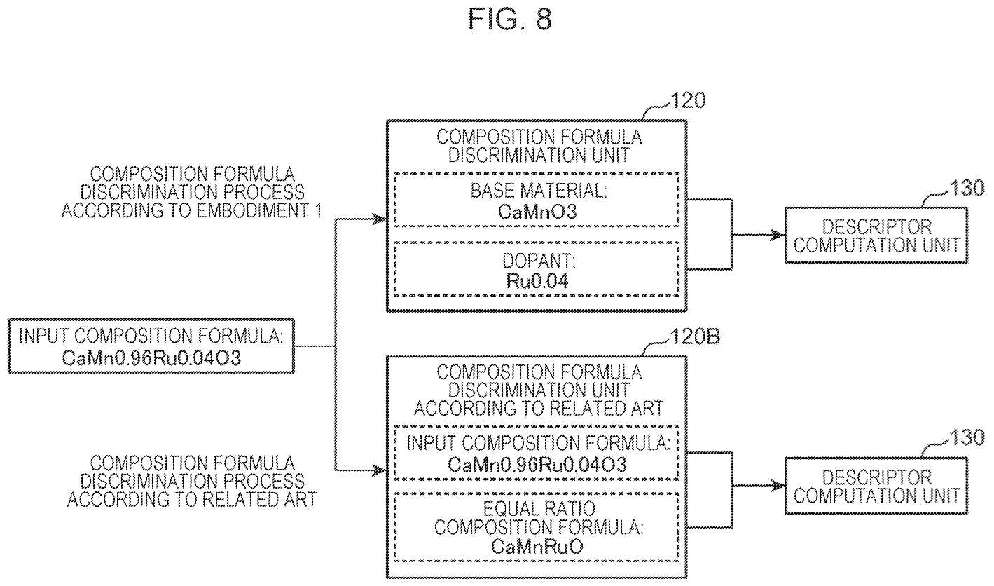

FIG. 8 is a schematic diagram for explaining specific differences between a composition formula discrimination process according to Embodiment 1 and a composition formula discrimination process according to the related art.

The composition formula discrimination unit 120 according to Embodiment 1 discriminates between a formula expressing a base material (CaMnO₃) and a dopant formula (Ru_{0.04}) forming the input composition formula (CaMn_{0.96}Ru_{0.04}O₃), and outputs the discriminated formula expressing the base material and a dopant list including the one or more dopant formulas to the descriptor computation unit 130. In contrast, a composition formula discrimination unit 120B according to the related art derives an equal ratio composition formula (CaMnRuO) from the input composition formula (CaMn_{0.96}Ru_{0.04}O₃), and outputs the input composition formula and the equal ratio composition formula to the descriptor computation unit 130.

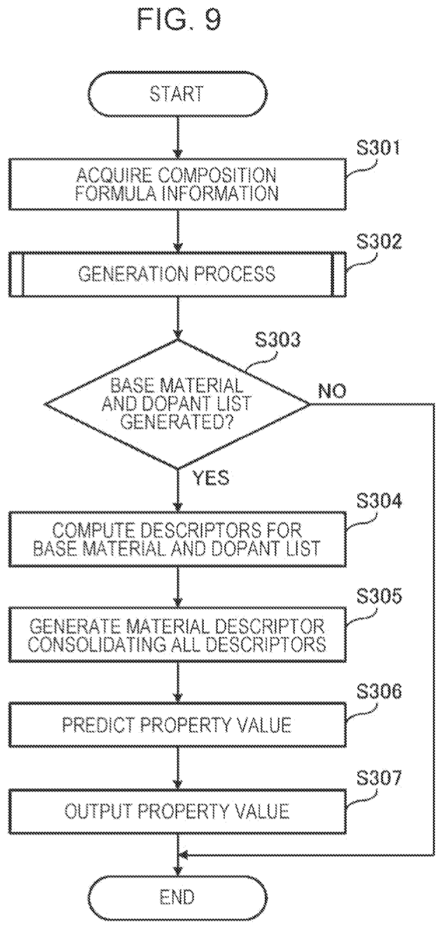

Next, FIG. 9 will be used to describe operations by the material property value prediction device 100 in Embodiment 1.

FIG. 9 is a flowchart for explaining operations by the material property value prediction device in Embodiment 1.

First, in step S301, the input acquisition unit 110 acquires an input composition formula from the input unit 210.

Next, in step S302, the composition formula discrimination unit 120 performs a generation process of generating a formula expressing the base material and a dopant list including one or more dopant formulas from the input composition formula. Details about the generation process will be described later.

Next, in step S303, the descriptor computation unit 130 determines whether or not the composition formula discrimination unit 120 has generated the formula expressing the base material and the dopant list including one or more dopant formulas. At this point, in the case of determining that the formula expressing the base material and the dopant list have not been generated, or in other words, in the case where a rejection label has been applied to the input composition formula (NO in step S303), the process ends.

In the case of determining that the formula expressing the base material and the dopant list have been generated (YES in step S303), in step S304, the descriptor computation unit 130 computes a descriptor for the formula expressing the base material and one or more descriptors for one or more formulas expressing the one or more dopants included in the dopant list. The descriptor computation unit 130 acquires a known parameter about the element included in each of the one or more formulas expressing the one or more dopants from the material information storage unit 221, and uses the acquired known parameter to compute or determine a descriptor expressing each dopant. In addition, the descriptor computation unit 130 acquires known parameters about each element included in the formula expressing the base material from the material information storage unit 221, and computes a weighted average of the acquired known parameters as the descriptor of the base material. In the case where the formula expressing the base material is CaMnO₃ and the average atomic volume is calculated as the descriptor, the descriptor computation unit 130 calculates {(atomic volume of Ca)+(atomic volume of Mn)+(atomic volume of O)×3}/5.

Note that in the case where the descriptor computation unit 130 acquires information needed to predict the property value in addition to the composition formula information, the descriptor computation unit 130 also computes or determines a descriptor for the information needed to predict the property value.

A single descriptor may be calculated or determined for a formula expressing a single dopant, or descriptors may be calculated or determined for a formula expressing a single dopant.

A single descriptor may be calculated for a formula expressing a single base material, or descriptors may be calculated for a formula expressing a single base material.

Next, in step S305, the descriptor consolidation unit 140 generates a material descriptor consolidating the descriptors computed by the descriptor computation unit 130.

At this time, the material descriptor may be a sequence obtained by concatenating all of the descriptors generated by the descriptor computation unit 130.

There may be one or more descriptors for the formula expressing a single base material included in the material descriptor. For example, in the case where the formula expressing a single base material is CaMnO₃ as illustrated in FIG. 30, the material descriptor of CaMnO₃ may include the average atomic volume of CaMnO₃ and the average density of CaMnO₃. Note that the average density of CaMnO₃ may be {(average density of Ca)+(average density of Mn)+(average density of O)×3}/5.

There may be one or more descriptors for the formula expressing a single dopant included in the material descriptor. For example, in the case where the formula expressing a single dopant is Ru_{0.04}, the material descriptor of Ru_{0.04} may include the atomic volume of Ru and/or the density of Ru.

Next, in step S306, the property value prediction unit 102 uses the material descriptor generated by the descriptor consolidation unit 140 to predict a property value of the material.

At this point, the predictive model used by the property value prediction unit 102 may include machine learning such as a neural network, a random forest, or a greedy algorithm, or an approximation according to a logical model formula.

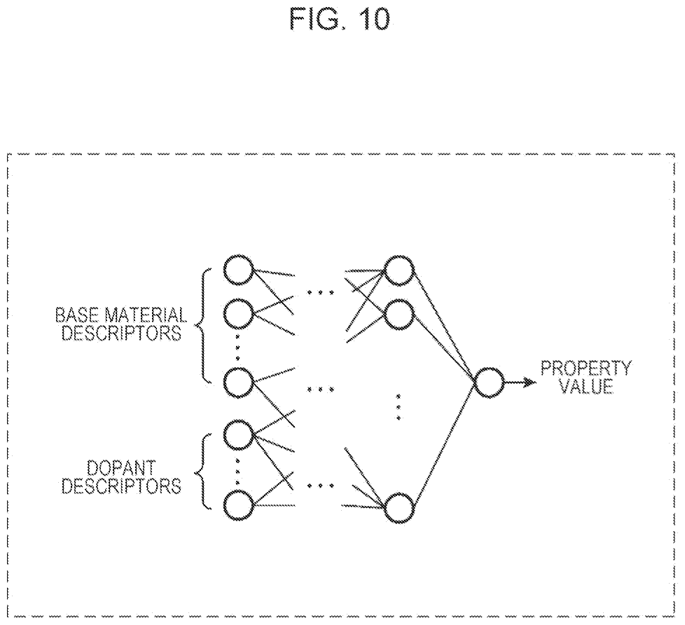

FIG. 10 is a diagram illustrating an example of property value prediction or machine learning by a neural network using base material descriptors and dopant descriptors. The property value prediction unit 102 inputs one or more descriptors with respect to a formula expressing a base material and one or more descriptors with respect to one or more formulas expressing one or more dopants into the units in the input layer of a predictive model, performs calculations based on the input signals and weight values in each unit included in the intermediate layer(s) and the output layer, and acquires a predetermined property value output from the unit in the output layer of the predictive model as a prediction result. In addition, the training unit 103 trains the predictive model by inputting one or more descriptors with respect to a formula expressing a base material and one or more descriptors with respect to one or more formulas expressing one or more dopants into the units in the input layer of the predictive model. It is sufficient to train the predictive model using training data that includes data sets containing predetermined property values corresponding to descriptors.

Returning to FIG. 9, next, in step S307, the output unit 230 outputs the predetermined property value predicted by the property value prediction unit 102.

Next, a specific example of the generation process in step S302 of FIG. 9 in Embodiment 1 will be described. The generation process in step S302 of FIG. 9 is different between the case where a base material list including formulas expressing base materials forming input composition formulas is stored in advance in the memory 220, and the case where the base material list is not stored in advance in the memory 220. Here, in the case where the composition formulas of the two materials “CaMn_{0.96}Ru_{0.4}O₃” and “Nb_{0.95}Ti_{0.05}FeSb” are included in the material information, for example, the base material list is a list of the formulas “CaMnO₃” and “NbFeSb” expressing the base materials of the two materials in advance. Note that a tag clearly indicating that the formula expressing the base material of the composition formula “CaMn_{0.96}Ru_{0.4}O₃” is “CaMnO₃” in the base material list may also be attached to the composition formula, for example.

In Embodiment 1, the memory 220 stores the base material list, and the generation process in step S302 of FIG. 9 is performed using the base material list.

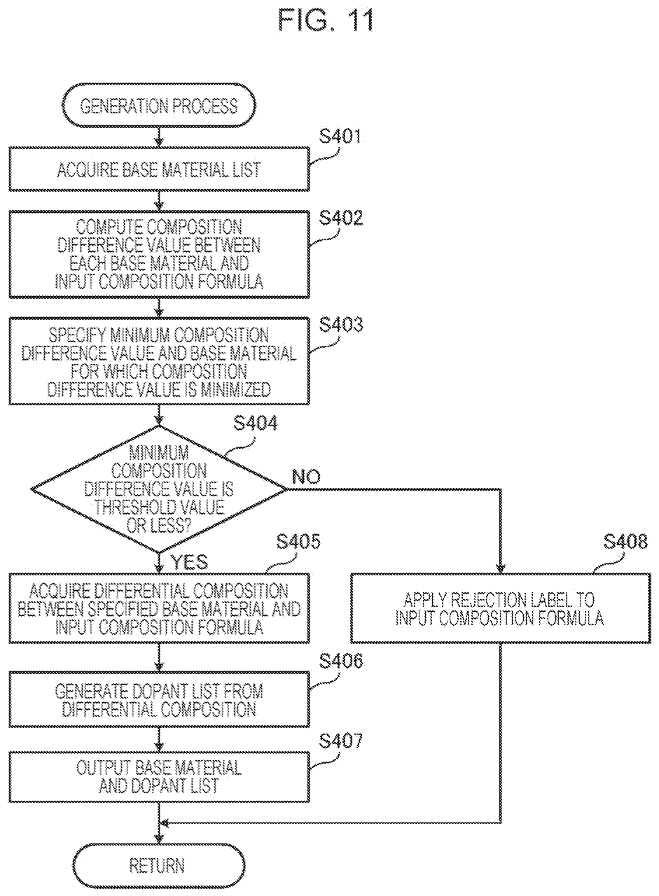

FIG. 11 will be used to describe the generation process in step S302 of FIG. 9 in Embodiment 1.

FIG. 11 is a flowchart for explaining the generation process in step S302 of FIG. 9 in Embodiment 1.

First, in step S401, the composition formula discrimination unit 120 acquires the base material list from the base material list storage unit 222. The description of base materials included in the base material list may include CaMnO₃.

Next, in step S402, the composition formula discrimination unit 120 computes a composition difference value between each of the formulas expressing the base materials included in the base material list and the input composition formula. Here, the composition difference value is the sum of the absolute values of the coefficients in the differential composition formula of the two composition formulas. For example, the differential composition formula between the formula “CaMnO₃” expressing the base material and the input composition formula “CaMn_{0.96}Ru_{0.4}O₃” is “Mn_{-0.4}Ru_{0.04}”, and the composition difference value is the sum of the absolute value of “−0.04” and the absolute value of “0.04”, or in other words “0.08”.

For example, the differential composition formula between the formula “CaMnO₃” expressing the base material and the input composition formula “CaMn_{0.95}Yb_{0.05}O₃” is “Mn_{-0.05}Yb_{0.05}”, and the composition difference value is the sum of the absolute value of “−0.05” and the absolute value of “0.05”, or in other words “0.10”.

The differential composition formula and the composition difference value may be defined as follows. Note that in the case where an atomic symbol included in a composition formula has a coefficient of 1, “1” is generally not indicated, but in the following description, cases where the coefficient is 1 will also be indicated. For example, the composition formula CaMnO₃ will be written as Ca₁Mn₁O₃.

Provided that A1, B1 . . . , A2, B2, and so on each represent an atomic symbol, a first (composition) formula is A1ₐ₁B1_{b1} . . . , and a second (composition) formula is A2ₐ₂B2_{b2} . . . , where A1≠A2 and B1≠B2, the differential (composition) formula between the first (composition) formula and the second (composition) formula is A2ₐ₂B2_{b2} . . . A1₋ₐ₁B1_{-b1} . . . , and the (composition) difference value between the first (composition) formula and the second (composition) formula is {|a2|+|b2|+ . . . +|−a1|+|−b1|+ . . . }. Note that A2ₐ₂, B2_{b2}, . . . , A1₋ₐ1, B1_{-b1}, and so on may be listed in any order.

In the case where A1=A2 and B1≠B2, the differential (composition) formula between the first (composition) formula and the second (composition) formula is A2₍ₐ₂₋ₐ₁₎B2_{b2} . . . B1_{-b1} . . . and the (composition) difference value between the first (composition) formula and the second (composition) formula is {|a2−a1|+|b2|+ . . . +|−b1|+ . . . }. Note that A2₍ₐ₂₋ₐ₁₎, B2_{b2}, and so on may be listed in any order.

In the case where A1=A2, B1≠B2, and a2=a1, the differential (composition) formula between the first (composition) formula and the second (composition) formula is B2_{b2} . . . B1_{-b1} . . . , and the (composition) difference value between the first (composition) formula and the second (composition) formula is {|b2|+ . . . +|−b1|+ . . . }. Note that B2_{b2}, . . . , B1_{-b1}, and so on may be listed in any order.

The differential composition formula and the composition difference value may also be defined as follows.

Let a 118-dimensional vector corresponding to each of the 118 existing elements be defined as the composition formula vector

\vec{v}

Let v_{A} denote the vector element corresponding to the element referred to as A in the composition formula vector. For example, v_{Mn} represents the vector element corresponding to Mn in the composition formula vector.

In the case of the composition formula vector for CaMnO₃, the numbers 1, 1, and 3 are input into v_{Ca}, v_{Mn}, and v_{O}, respectively, while 0 is input into the remaining vector elements. This composition formula vector for CaMnO₃ is denoted

\vec{v}(CaMnO₃)

When given two composition formulas c1 and c2, the differential vector

\vec{v}=\vec{v} (c1)

\vec{v} (c2)

of the composition formula vectors corresponding to the composition formulas is introduced.

At this point, let the sum of the absolute values of all vector elements in the differential vector be a composition difference value d:

d=Σ(|\vec{v′}ᵢ|)

Also, for all elements whose corresponding vector element is non-zero, let a composition formula in which the corresponding vector element values are arranged as coefficients be the differential composition formula. For example, provided that

\vec{v′}=\vec{v}(CaMn_{0.96}Ru_{0.04}O₃)−\vec{v}(CaMnO₃)

the differential vector is a 118-dimensional vector in which v′_{Mn}=−0.04, v′_{Ru}=0.04, and all other vector elements are 0, the composition difference value is d=|−0.04|+|0.04|=0.08, and the differential composition formula is Mn_{-0.04}Ru_{0.04} in which Mn having a coefficient of −0.04 and Ru having a coefficient of 0.04 are arranged. The elements in the differential composition formula may be written in any order. Note that in the case where the composition difference value is 0, a differential composition formula does not exist.

Next, in step S403, the composition formula discrimination unit 120 specifies a minimum composition difference value and a formula expressing the base material used to obtain the minimum composition difference value from among the composition difference values. For example, in the case where the input composition formulas are “CaMn_{0.96}Ru_{0.04}O₃” and “CaMn_{0.95}Yb_{0.05}O₃”, the minimum composition difference value is “0.08”. As described with regard to step S402, the composition difference value (0.08) associated with CaMn_{0.96}Ru_{0.04}O₃ is smaller than the composition difference value (0.10) associated with CaMn_{0.95}Yb_{0.05}O₃.

Next, in step S404, the composition formula discrimination unit 120 determines whether or not the minimum composition difference value is a threshold value or less. At this point, in the case of determining that the minimum composition difference value is the threshold value or less (YES in step S404), in step S405, the composition formula discrimination unit 120 acquires a differential composition formula between the formula expressing the base material used to obtain the minimum composition difference value being the threshold value or less and the input composition formula. In the case of the above example, the composition formula discrimination unit 120 acquires the differential composition formula “Mn_{-0.04}Ru_{0.04}”. This is because 0.08 (the composition difference value of the differential composition formula “Mn_{-0.04}Ru_{0.04}”)<0.10 (the composition difference value of the differential composition formula “Mn_{-0.05}Yb_{0.05}”).

Next, in step S406, the composition formula discrimination unit 120 generates a dopant list including one or more formulas expressing one or more dopants from the differential composition formula. For example, in the case where the differential composition formula is “Mn_{-0.4}Ru_{0.04}”, the dopant list, includes the formula “Ru_{0.04}” expressing the dopant, but does not have to include the formula “Mn_{-0.04}” expressing the host (that is, the element that is doped). The dopant list may include both the formula “Ru_{0.04}” expressing the dopant and the formula “Mn_{-0.04}” expressing the host. In the differential composition formula, a positive coefficient is associated with a dopant, while a negative coefficient is associated with a host.

Next, in step S407, the composition formula discrimination unit 120 outputs information including the formula expressing the base material specified in step S403 and the dopant list generated in step S406 to the descriptor computation unit 130.

On the other hand, in the case where the minimum composition difference value is determined to be greater than the threshold value in step S404 (NO in step S404), in step S408, the composition formula discrimination unit 120 applies a rejection label to the input composition formula.

Note that in the case where the descriptor consolidation unit 140 acquires information that may influence the material property value from the material information storage unit 221, such as information about the structure of the material and/or information about the test environment of the material, the descriptor consolidation unit 140 may also generate a material descriptor in which one or more descriptors derived from the information that may influence the material property value and descriptors computed from the input composition formula are consolidated into a single sequence. The information about the structure of the material is information such as a parameter derived using three-dimensional position information about each element included in the input composition formula of the material, or a parameter derived using information about the position of each element included in the input composition formula of the material, for example. Also, the information about the test environment of the material is information such as information about the temperature when the material is generated, information about the temperature when the property of the material is measured, or a specific method of generating the material, for example. A parameter obtained by performing a first-principles calculation using information about the three-dimensional positions of the elements included in the base material included in the material composition formula, such as the band gap and/or the effective mass, may also be adopted as a descriptor.

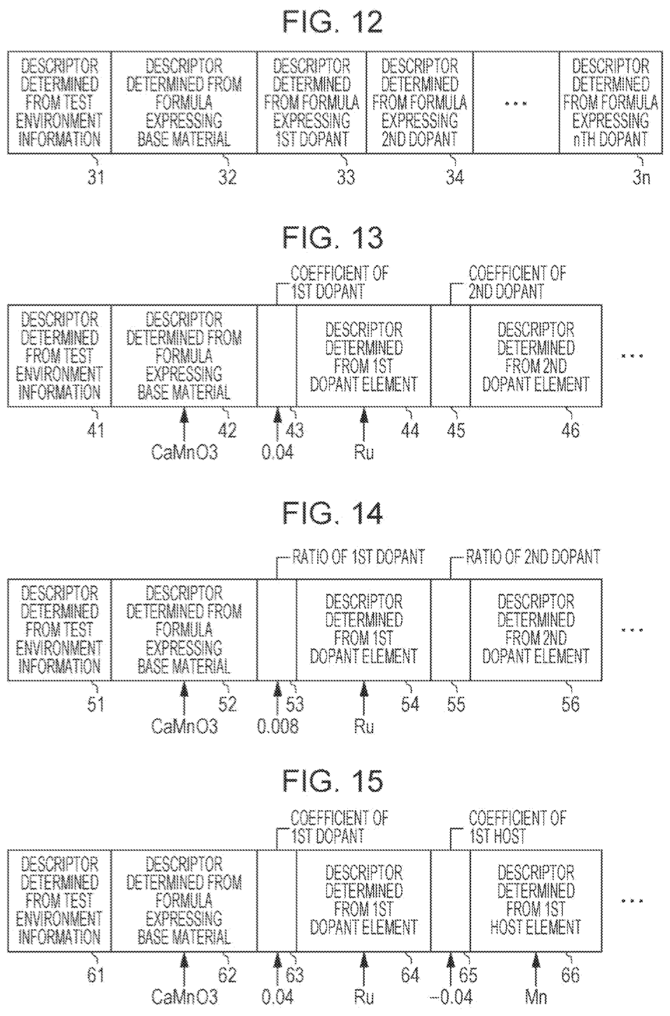

FIG. 12 is a diagram illustrating an example of a material descriptor including a descriptor computed from test environment information. In FIG. 12, a descriptor 31 computed from the test environment information, a descriptor 32 computed from the formula expressing the base material, and descriptors 33 to 3n computed from formulas respectively expressing 1st to nth dopants are arranged to form a single material descriptor. The descriptor 31 computed from the test environment information may be one or more descriptors.

Note that the input acquisition unit 110 may also acquire test environment information indicating the environment where the material is generated. The descriptor computation unit 130 may compute a descriptor corresponding to the formula expressing the base material, one or more descriptors corresponding to one or more formulas expressing one or more dopants included in the dopant list, and a descriptor corresponding to the test environment information.

In the prediction mode, the user may use the input unit 210 to input test environment information indicating the environment where the material corresponding to the input composition formula of the material is generated. The input acquisition unit 110 may acquire the test environment information indicating the environment where the material is generated from the input unit 210, and forward the information to the descriptor computation unit 130 and the material information storage unit 221. The material information storage unit 221 may store the information.

In the training mode, the material information storage unit 221 may store in advance test environment information indicating environments where materials corresponding to the composition formulas of materials are generated, respectively. The input acquisition unit 110 may acquire the test environment information indicating the environments where materials are generated from the material information storage unit 221, and forward the information to the descriptor computation unit 130.

The input acquisition unit 110 may also acquire information indicating the structure of the material. The descriptor computation unit 130 may compute a descriptor corresponding to the formula expressing the base material, one or more descriptors corresponding to one or more formulas expressing one or more dopants included in the dopant list, and a descriptor corresponding to the structure information.

In the prediction mode, the user may use the input unit 210 to input structure information indicating the structure of the material corresponding to the input composition formula of the material. The input acquisition unit 110 may acquire the structure information indicating the structure of the material from the input unit 210, and forward the information to the descriptor computation unit 130 and the material information storage unit 221. The material information storage unit 221 may store the information.

In the training mode, the material information storage unit 221 may store in advance structure information indicating the structures of the materials corresponding to the composition formulas of the materials. The input acquisition unit 110 may acquire the structure information indicating the structures of the materials from the material information storage unit 221, and forward the information to the descriptor computation unit 130.

Note that the descriptors included in the material descriptor generated by the descriptor computation unit 130 may also include a descriptor indicating the coefficient of an atomic symbol included in the formula expressing a dopant. The descriptor computation unit 130 may also add the coefficient of an atomic symbol included in the formula expressing a dopant included in the dopant list to the material descriptor as a descriptor.

FIG. 13 is a diagram illustrating an example of a material descriptor including descriptors indicating coefficients of atomic symbols included in formulas expressing dopants. FIG. 13 illustrates an example of a material descriptor computed from the input composition formula CaMn_{0.96}Ru_{0.04}O₃. A descriptor 43 illustrated in FIG. 13 expresses the coefficient 0.04 of the atomic symbol Ru included in the formula “Ru_{0.04}” expressing a 1st dopant. The coefficient of an atomic symbol included in the formula expressing each dopant is placed immediately before the descriptor computed from the formula expressing each dopant.

The descriptor computation unit 130 may also compute the ratio of the coefficient of the atomic symbol included in the formula expressing the dopant with respect to the sum of the coefficients of all atomic symbols included in the composition formula, and include a descriptor indicating the computed ratio in the material descriptor.

FIG. 14 is a diagram illustrating an example of a material descriptor including a descriptor that indicates a ratio of the coefficient of an atomic symbol included in the composition formula of a dopant with respect to the sum of the coefficients of all atomic symbols included in the input composition formula. FIG. 14 illustrates an example of a material descriptor computed from the input composition formula CaMn_{0.96}Ru_{0.4}O₃. A descriptor 53 illustrated in FIG. 14 indicates the ratio of the coefficient of an atomic symbol included in a formula expressing a 1st dopant with respect to the sum of the coefficients of all atomic symbols included in the input composition formula. The descriptor 53 expresses the value 0.008, which is obtained by dividing the coefficient 0.04 of the 1st dopant, namely Ru, by the sum 5 of the coefficients of all atomic symbols included in the input composition formula. The ratio of the coefficient of an atomic symbol included in the composition formula of a dopant with respect to the sum of the coefficients of all atomic symbols included in the input composition formula may also be referred to as the ratio of the dopant. The descriptor indicating the ratio of the dopant is placed immediately before the descriptor computed from the formula expressing the dopant.

The descriptors included in the material descriptor generated by the descriptor computation unit 130 may also include a descriptor indicating the coefficient of an atomic symbol included in the formula expressing a host. For example, when comparing the input composition formula CaMn_{0.96}Ru_{0.04}O₃ to the base material CaMnO₃, the host refers to Mn whose ratio is reduced by the doping with Ru_{0.04}. The descriptor computation unit 130 may also add the coefficient of a host whose ratio is reduced by the doping with one or more dopants included in the dopant list as a descriptor.

FIG. 15 is a diagram illustrating an example of a material descriptor including a coefficient of a host. FIG. 15 illustrates an example of a material descriptor computed from the input composition formula CaMn_{0.96}Ru_{0.04}O₃, in which the base material composition formula is CaMnO₃. At this time, Ru_{0.04} is the dopant with an addition of 0.04, while Mn_{0.96} is the host with a subtraction of 0.04. The descriptor indicating the coefficient of the host illustrated in FIG. 15 describes this “subtraction of 0.04” as an “addition of −0.04”. A descriptor 63 illustrated in FIG. 15 expresses the coefficient 0.04 of the formula “Ru_{0.04}” that expresses the 1st dopant, while a descriptor 65 expresses the coefficient −0.04 of the formula “Mn_{0.96}”, or in other words “Mn_{-0.04}”, that expresses the first host. In the descriptor 63, the coefficient of the 1st dopant Ru is expressed using a positive sign, whereas in the descriptor 65, the coefficient of the host Mn is expressed using a negative sign. The descriptor indicating the coefficient of the formula expressing the host is placed immediately before or immediately after the descriptor computed from the formula expressing the host.

Note that in the case where material descriptors calculated from different composition formulas have different lengths, the material descriptors may be set to the same length. In other words, a material descriptor calculated from a composition formula may be set to a fixed length. This is so that even if the number of formulas expressing dopants computed from a composition formula is different from the number of formulas expressing dopants computed from a composition formula, the material descriptor computed from the former composition formula and the material descriptor computed from the latter composition formula can be contained in a single database. The material descriptors contained in the database are used by predictive models having the same number of input units, for example.

Hereinafter, a method of setting a material descriptor to a fixed length will be described.

In the case where the descriptor consolidation unit 140 does not receive a predetermined number of descriptors calculated or determined from formulas expressing dopants from the descriptor computation unit 130, the descriptor consolidation unit 140 places zero or an average value in a predetermined location of the material descriptor. Note that the average value will be described later. The predetermined number is a natural number n equal to or greater than 2 for example, and may be a maximum number from among numbers. Each of the numbers is a number of formulas expressing dopants derived from each of the input composition formulas being to be acquired. For example, the number of the formulas expressing dopants derived from the input composition formula “CaMn_{0.96}Ru_{0.4}O₃” is one and the formula expressing the dopant is Ru_{0.04}, and the number of the formulas expressing dopants derived from the input composition formula “Ca_{0.9}Bi_{0.1}Mn_{0.9}Nb_{0.1}O₃” is two and the formulas expressing the dopants are Bi_{0.1} and Nb_{0.1}. A first material descriptor computed from the input composition formula “Ca_{0.9}Bi_{0.1}Mn_{0.9}Nb_{0.1}O₃” includes a first descriptor and a second descriptor computed or determined from the two formulas expressing the two dopants. The first descriptor is placed in a first location of the first material descriptor, and the second descriptor is placed in a second location of the first material descriptor.

A second material descriptor computed from the input composition formula “CaMn_{0.96}Ru_{0.04}O₃” includes a third descriptor computed or determined from the single formula expressing the single dopant. The third descriptor is placed in a third location of the second material descriptor, and zero or an average value is placed in a fourth location of the second material descriptor.

The first material descriptor and the second material descriptor are the same length. The first location in the first material descriptor and the third location in the second material descriptor may be at the same position in a structure of the material descriptor, and the second location in the first material descriptor and the fourth location in the second material descriptor may be at the same position in the structure of the material descriptor. Alternatively, the first location in the first material descriptor and the fourth location in the second material descriptor may be at the same position in the structure of the material descriptor, while in addition, the second location in the first material descriptor and the third location in the second material descriptor may be at the same position in the structure of the material descriptor.

With this arrangement, it is possible to train a predictive model using material descriptors as a single database without losing information.

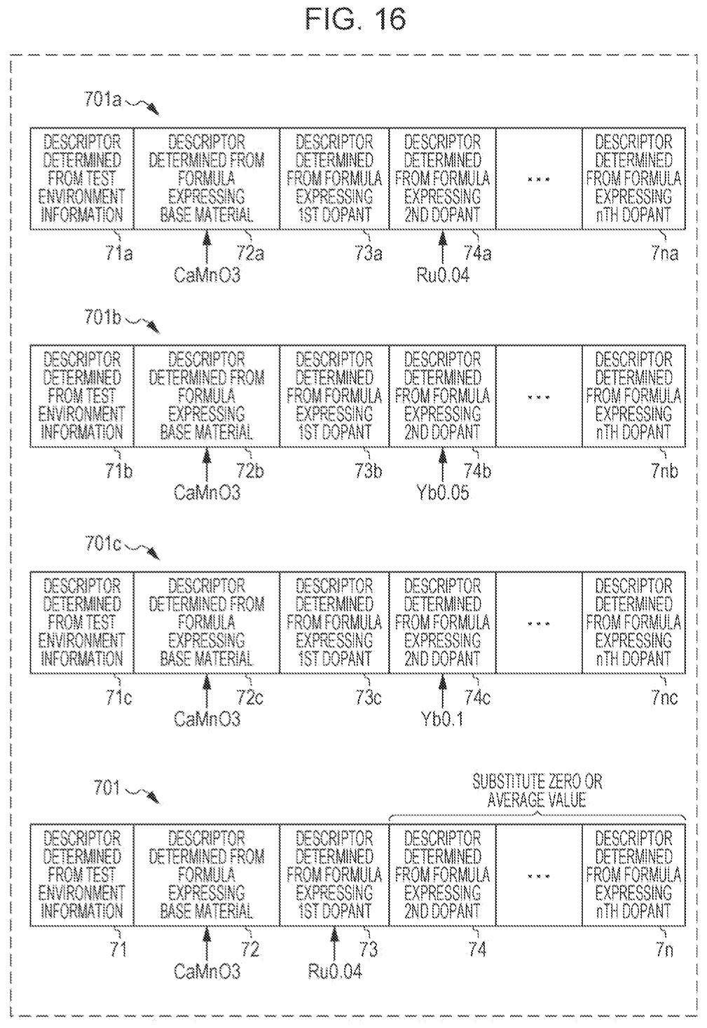

FIG. 16 is a diagram illustrating an example of a material descriptor in which zero or an average value is placed in a location where a descriptor calculated or determined from a formula expressing a dopant should be placed. As illustrated in FIG. 16, in a material descriptor 701, because a 1st dopant exists, a descriptor 73 computed from the formula expressing the 1st dopant exists, but because 2nd to nth dopants do not exist, zero or an average value is placed in each of the locations where descriptors 74 to 7n respectively computed from formulas expressing the 2nd to nth dopants should be placed. Note that in the case where an ith dopant does not exist in a first material descriptor, the descriptor computation unit 130 may adopt an average value of the descriptors for existing dopants among the ith dopant in a 2nd material descriptor to the ith dopant in an nth material descriptor as the descriptor of the ith dopant of the first material descriptor. The descriptors of the ith dopant in the 1st material descriptor to the ith dopant in the nth material descriptor exist at the same positions from a data structure perspective.

For example, in FIG. 16, an average value of the descriptors 74a to 74c computed from the second dopants in material descriptors 701a to 701c is placed in the descriptor 74 of the material descriptor 701.

Additionally, in the case where zero or an average value is placed in the portion where a descriptor computed from a formula expressing a dopant in a material descriptor is to be placed, the position in the material descriptor of that portion may be a location where a descriptor computed from a formula expressing another dopant is placed.

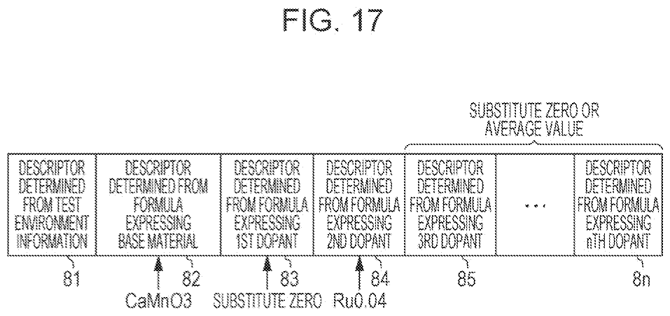

FIG. 17 is a diagram illustrating another example of a material descriptor in which zero or an average value is placed in a location where a descriptor calculated or determined from a formula expressing a dopant should be placed. As illustrated in FIG. 17, the input composition formula contains the formula Ru_{0.04} expressing a single dopant, but a descriptor computed or determined from the formula expressing the dopant may be placed not at the position where a descriptor 83 computed or determined from the formula expressing the 1st dopant is placed, but instead at the position where a descriptor 84 computed or determined from a formula expressing a second dopant is placed. Additionally, zero or an average value may be placed at the position where the descriptor 83 computed or determined from the formula expressing the 1st dopant is placed, and zero or an average value may be placed at the positions where descriptors 85 to 8n computed from formulas expressing 3rd to nth dopants are placed.

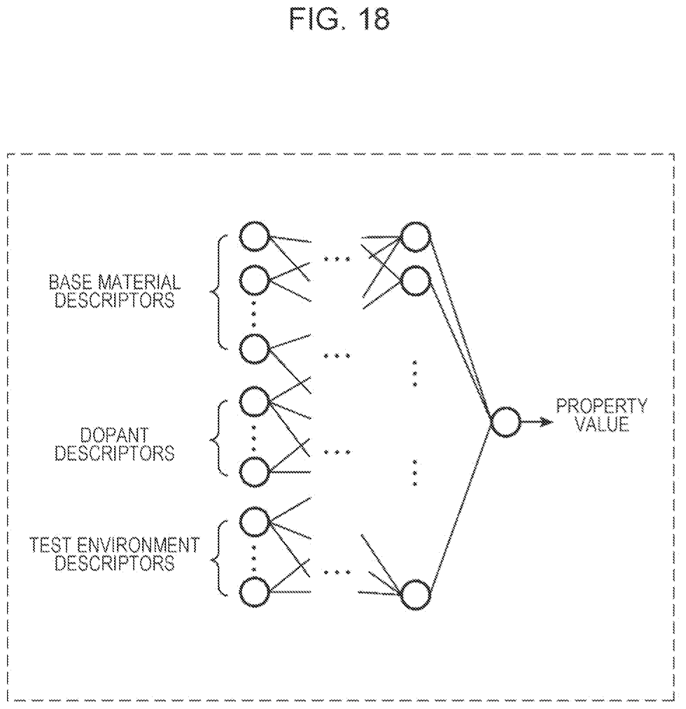

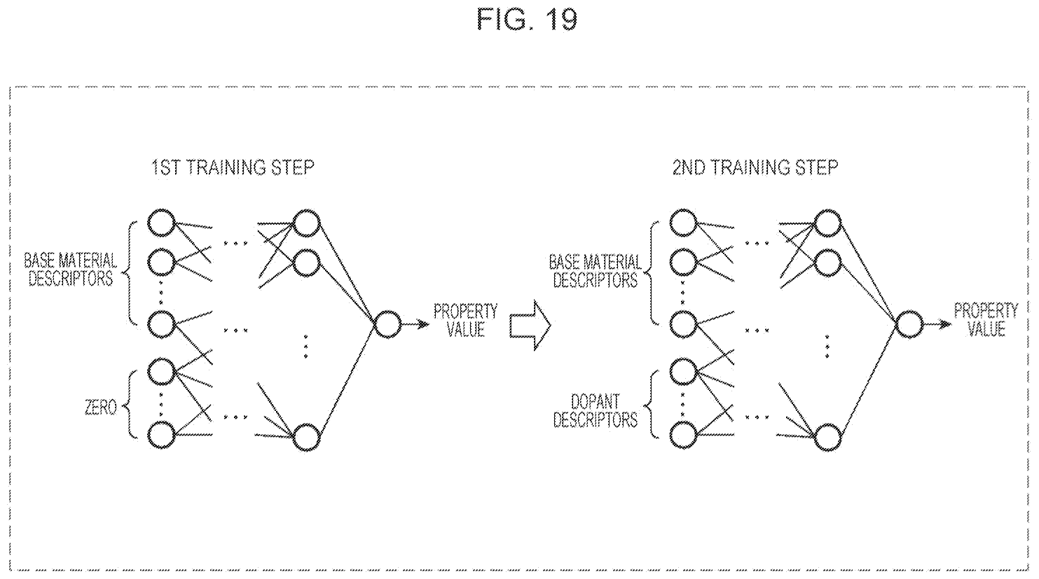

Note that in the case of using the test environment descriptor, the test environment descriptor may also be input into the predictive model together with the base material descriptor and the dopant descriptor, as illustrated in FIG. 18.

FIG. 18 is a diagram illustrating an example of property value prediction or machine learning by a neural network using base material descriptors, dopant descriptors, and test environment descriptors. As illustrated in FIG. 18, the property value prediction unit 102 inputs one or more descriptors with respect to a formula expressing a base material, one or more descriptors with respect to one or more formulas expressing one or more dopants, and one or more descriptors of one or more test environments into the units in the input layer of a predictive model, and acquires a predetermined property value output from the unit in the output layer of the predictive model as a prediction result. In addition, the training unit 103 trains the predictive model by inputting one or more descriptors with respect to a formula expressing a base material, one or more descriptors with respect to one or more formulas expressing one or more dopants, and one or more descriptors of one or more test environments into the units in the input layer of the predictive model. Note that in FIG. 18, not only the test environment descriptor but also one or more descriptors of one or more pieces of structure information may be input into the predictive model together with one or more descriptors with respect to a formula expressing a base material and one or more descriptors with respect to one or more formulas expressing one or more dopants. It is sufficient to train the predictive model using training data that includes data sets containing predetermined property values corresponding to descriptors.

Note that in Embodiment 1, the training unit 103 may also perform multilevel training including a first training step that trains the predictive model by using the base material descriptor without using the dopant descriptor, and a second training step that trains the predictive model by using both the base material descriptor and the dopant descriptor.

FIG. 19 is a diagram illustrating an example of multilevel machine learning by a neural network using base material descriptors and dopant descriptors. As illustrated in FIG. 19, in the first training step, the training unit 103 trains the neural network by using base material descriptors without using dopant descriptors, while in the second training step, the training unit 103 trains the neural network by using base material descriptors and dopant descriptors. Note that a third training step using test environment descriptors and structure information descriptors may also be added as a level in the same way to train the neural network in a multilevel way.

### Embodiment 2

In Embodiment 1, the memory 220 stores the base material list, but in Embodiment 2, the memory 220 does not store the base material list.

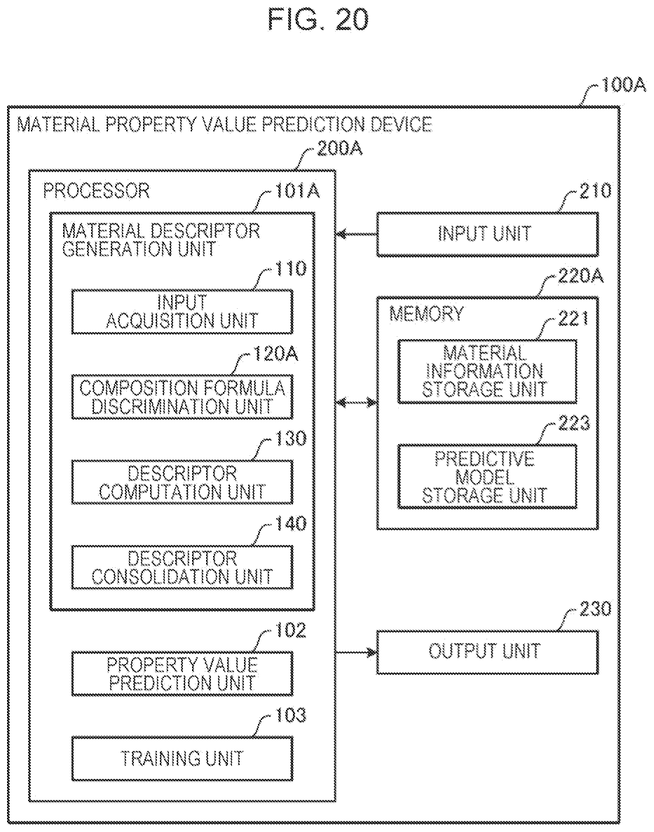

FIG. 20 is a diagram illustrating a configuration of a material property value prediction device in Embodiment 2. A material property value prediction device 100A in Embodiment 2 includes a processor 200A, an input unit 210, memory 220A, and an output unit 230. The processor 200A includes a material descriptor generation unit 101A, a property value prediction unit 102, and a training unit 103. Additionally, the material descriptor generation unit 101A includes an input acquisition unit 110, a composition formula discrimination unit 120A, a descriptor computation unit 130, and a descriptor consolidation unit 140. The memory 220A includes a material information storage unit 221 and a predictive model storage unit 223. Note that in Embodiment 2, components that are the same as Embodiment 1 are denoted with the same signs, and description of such components will be omitted.

The composition formula discrimination unit 120A selects an atomic symbol and its coefficient from the input composition formula acquired from the input acquisition unit 110. The composition formula discrimination unit 120A determines whether or not the coefficient is greater than a threshold value. In the case of determining that the coefficient is the threshold value or less, the composition formula discrimination unit 120A adds the atomic symbol to the dopant list. In the case of determining that the coefficient is greater than the threshold value, the composition formula discrimination unit 120A adds the combination of the atomic symbol and a new coefficient generated by rounding up the fractional part of the coefficient to a base material element list. After performing the above process on all atomic symbols included in the input composition formula, the composition formula discrimination unit 120A derives a formula expressing the base material that consolidates the elements included in the base material element list, or in other words, the “combinations of an atomic symbol and a new coefficient generated by rounding up the fractional part of the coefficient”. The composition formula discrimination unit 120A outputs the base material and the dopant list.

The operations by the material property value prediction device 100A in Embodiment 2 are the same as the operations by the material property value prediction device 100 in Embodiment 1 illustrated in FIG. 9, and therefore a description is omitted. The operation that is different between Embodiment 2 and Embodiment 1 is the generation process in step S302 of FIG. 9.

In Embodiment 2, because the memory 220 does not store the base material list, the generation process in step S302 of FIG. 9 is performed without using the base material list.

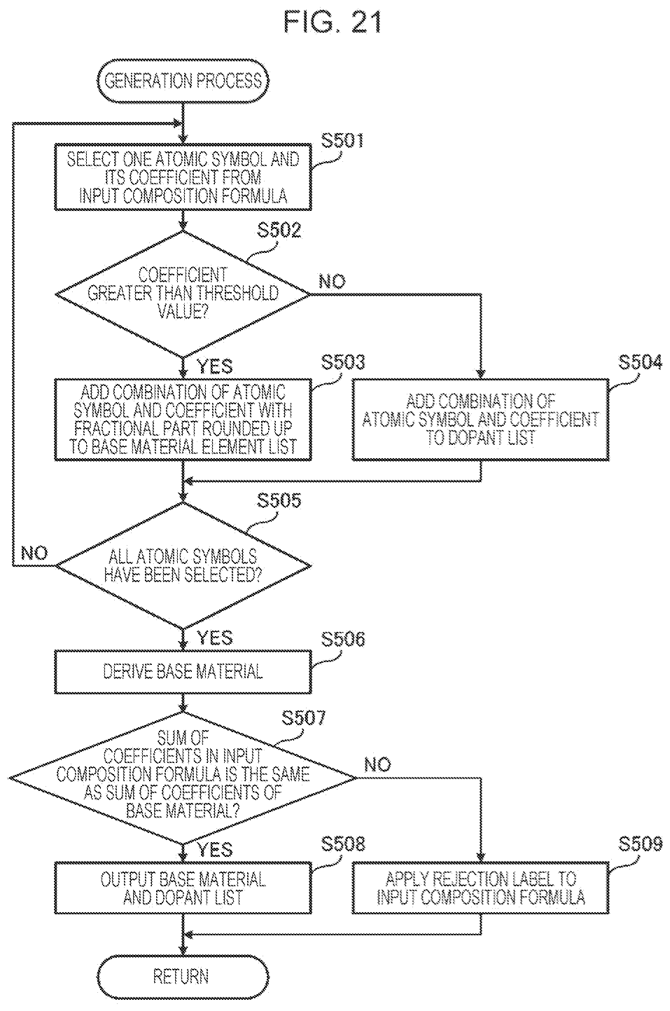

FIG. 21 will be used to describe the generation process in step S302 of FIG. 9 in Embodiment 2.

FIG. 21 is a flowchart for explaining the generation process in step S302 of FIG. 9 in Embodiment 2.

First, in step S501, the composition formula discrimination unit 120A selects an atomic symbol and its coefficient from the input composition formula.

Next, in step S502, the composition formula discrimination unit 120A determines whether or not the selected coefficient is greater than a threshold value. Note that the threshold value is 0.5, for example. At this point, in the case of determining that the coefficient is greater than the threshold value (YES in step S502), in step S503, the composition formula discrimination unit 120A adds the combination of the selected atomic symbol and a new coefficient generated by rounding up the fractional part of the coefficient to the base material element list. For example, in the case where the atomic symbol is Mn and the coefficient of the atomic symbol is 0.96, rounding up the fractional part results in a new coefficient of 1, and “Mn₁” is added to the base material element list. Note that in the case where the coefficient of the atomic symbol is 1.5, rounding up the fractional part results in a new coefficient of 2.

On the other hand, in the case of determining that the coefficient is the threshold value or less (NO in step S502), in step S504, the composition formula discrimination unit 120A adds the combination of the selected atomic symbol and the selected coefficient to the dopant list.

Next, in step S505, the composition formula discrimination unit 120A determines whether or not all atomic symbols included in the input composition formula have been selected. At this point, in the case of determining that not all atomic symbols have been selected (NO in step S505), the process returns to step S501.

On the other hand, in the case of determining that all atomic symbols have been selected (YES in step S505), in step S506, the composition formula discrimination unit 120A derives the formula expressing the base material by consolidating the elements included in the base material element list, or in other words, the “combinations of an atomic symbol and a new coefficient generated by rounding up the fractional part of the coefficient”. For example, in the case where the base material element list is [Ca₁, Mn₁, O₃], the concatenation “CaMnO₃” of all elements in the base material element list is derived as the formula expressing the base material.

Next, in step S507, the composition formula discrimination unit 120A determines whether or not the sum of the coefficients in the input composition formula is the same as the sum of the coefficients in the formula expressing the base material.

At this point, in the case of determining that the sum of the coefficients in the input composition formula is the same as the sum of the coefficients in the formula expressing the base material (YES in step S507), in step S508, the composition formula discrimination unit 120A outputs the formula expressing the base material and the dopant list to the descriptor computation unit 130.

For example, in the case where the input composition formula is CaMn_{0.96}Ru_{0.04}O₃, and the formula expressing the base material is derived as CaMnO₃, (sum of coefficients in input composition formula)=(1+0.96+0.04+3)=5, and (sum of coefficients in formula expressing base material)=(1+1+3)=5.

On the other hand, in the case of determining that the sum of the coefficients in the input composition formula is different from the sum of the coefficients in the formula expressing the base material (NO in step S507), in step S509, the composition formula discrimination unit 120A applies a rejection label to the input composition formula.

Note that in Embodiment 2, the composition formula discrimination unit 120A does not have to perform the determination process in step S507. In this case, after deriving the formula expressing the base material in step S506, the composition formula discrimination unit 120A may output the formula expressing the base material and the dopant list to the descriptor computation unit 130 in step S508.

Note that the composition formula discrimination unit 120A may also send the formula expressing the base material to the memory 220A, and the memory 220A may record the formula expressing the base material. The process described in Embodiment 2 above may be performed on input composition formulas, formulas expressing base materials may be recorded in the memory 220A, and a base material list containing the recorded formulas expressing base materials may be generated. The generated base material list may be used as the base material list described in Embodiment 1.

### Embodiment 3

In Embodiment 1, the memory 220 stores the base material list. In Embodiment 3, a formula expressing the base material is derived by a discrimination process similar to Embodiment 2, and it is confirmed whether the derived formula expressing the base material exists in the base material list.

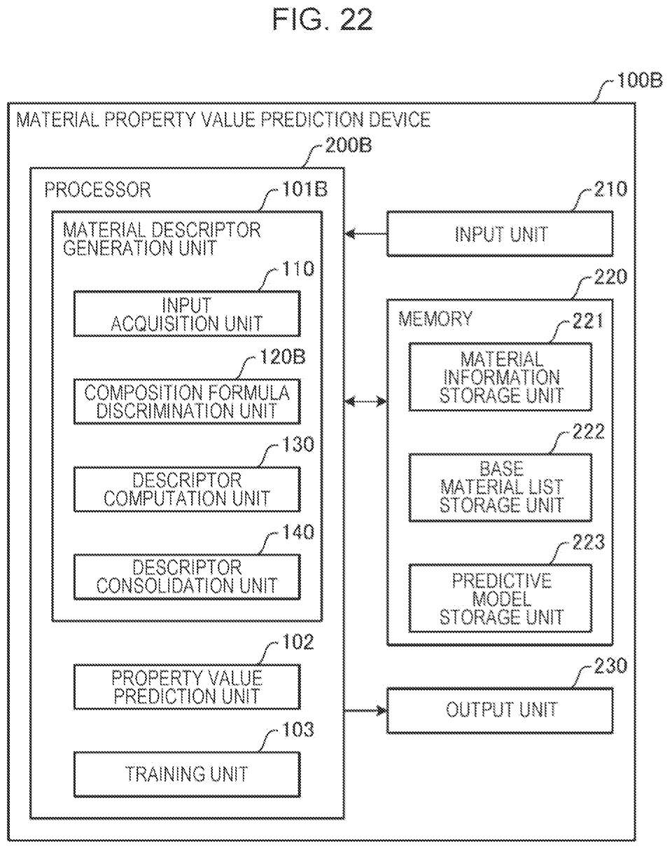

FIG. 22 is a diagram illustrating a configuration of a material property value prediction device in Embodiment 3. A material property value prediction device 100B in Embodiment 3 includes a processor 200B, an input unit 210, memory 220, and an output unit 230.

The processor 200B includes a material descriptor generation unit 101B, a property value prediction unit 102, and a training unit 103. Additionally, the material descriptor generation unit 101B includes an input acquisition unit 110, a composition formula discrimination unit 120B, a descriptor computation unit 130, and a descriptor consolidation unit 140. The memory 220 includes a material information storage unit 221, a base material list storage unit 222, and a predictive model storage unit 223. Note that in Embodiment 3, components that are the same as Embodiment 1 are denoted with the same signs, and description of such components will be omitted.

The composition formula discrimination unit 120B acquires a base material list including formulas expressing base materials from the base material list storage unit 222. The composition formula discrimination unit 120B determines whether or not the sum of the coefficients of the atomic symbols in the input composition formula acquired from the input acquisition unit 110 is an integer. In the case of determining that the sum of the coefficients of the atomic symbols in the input composition formula is an integer, the composition formula discrimination unit 120B selects an atomic symbol and its coefficient from the input composition formula. The composition formula discrimination unit 120B determines whether or not the coefficient is greater than a threshold value. In the case of determining that the coefficient is the threshold value or less, the composition formula discrimination unit 120B adds the element to the dopant list. In the case of determining that the coefficient is greater than the threshold value, the composition formula discrimination unit 120B adds the combination of the atomic symbol and a new coefficient generated by rounding up the fractional part of the coefficient to a base material element list.

After performing the above process on all atomic symbols included in the composition formula, the composition formula discrimination unit 120B derives a formula expressing the base material that consolidates the elements included in the base material element list, or in other words, the “combinations of an atomic symbol and a new coefficient generated by rounding up the fractional part of the coefficient”. The composition formula discrimination unit 120B determines whether or not the derived formula expressing the base material exists in the base material list. In the case of determining that the formula expressing the base material exists in the base material list, the composition formula discrimination unit 120B outputs the formula expressing the base material and the dopant list. In the case of determining that the sum of the coefficients in the input composition formula is not an integer, or in the case of determining that the formula expressing the base material does not exist in the base material list, the composition formula discrimination unit 120B applies a rejection label to the input composition formula.

The operations by the material property value prediction device 100B in Embodiment 3 are the same as the operations by the material property value prediction device 100 in Embodiment 1 illustrated in FIG. 9, and therefore a description is omitted. The operation that is different between Embodiment 3 and Embodiment 1 is the generation process in step S302 of FIG. 9.

In Embodiment 3, because the memory 220 stores the base material list, the generation process in step S302 of FIG. 9 is performed using the base material list.

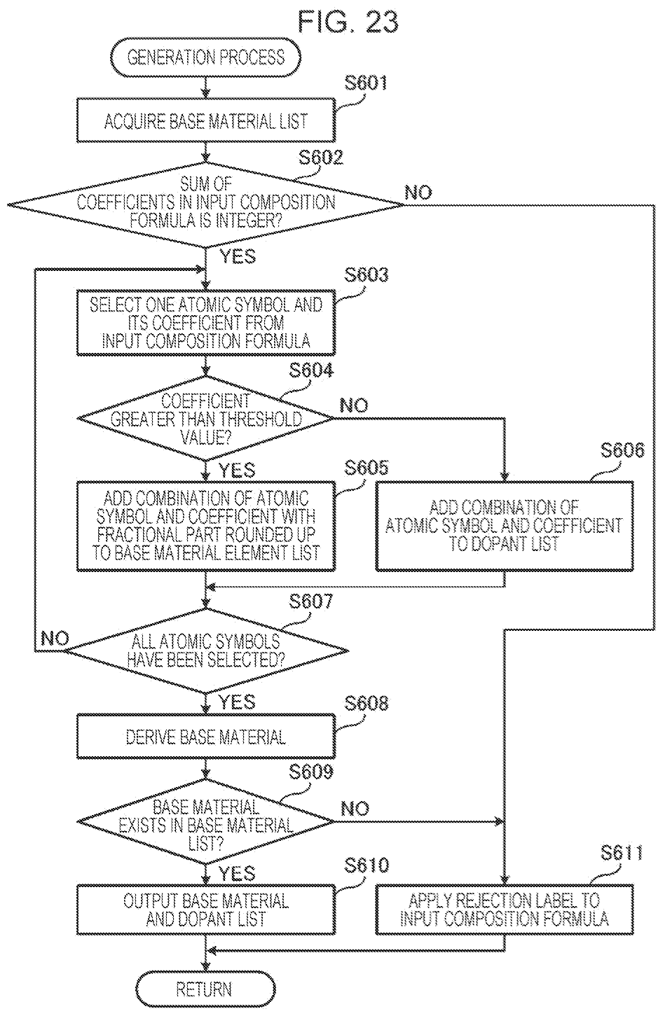

FIG. 23 will be used to describe the generation process in step S302 of FIG. 9 in Embodiment 3.

FIG. 23 is a flowchart for explaining the generation process in step S302 of FIG. 9 in Embodiment 3.

First, in step S601, the composition formula discrimination unit 120B acquires the base material list from the base material list storage unit 222.

Next, in step S602, the composition formula discrimination unit 120B determines whether or not the sum of the coefficients of the atomic symbols included in the input composition formula is an integer. This determination is made to set a material that is clearly known to be a host corresponding to a dopant as the target of generation. At this point, in the case of determining that the sum of the coefficients in the input composition formula is not an integer (NO in step S602), the process proceeds to step S611.

On the other hand, in the case of determining that the sum of the coefficients in the input composition formula is an integer (YES in step S602), in step S603, the composition formula discrimination unit 120B selects an atomic symbol and its coefficient from the input composition formula.

Next, in step S604, the composition formula discrimination unit 120B determines whether or not the selected coefficient is greater than a threshold value. Note that the threshold value is 0.5, for example. At this point, in the case of determining that the coefficient is greater than the threshold value (YES in step S604), in step S605, the composition formula discrimination unit 120B adds the combination of the selected atomic symbol and a new coefficient generated by rounding up the fractional part of the coefficient to the base material element list.

On the other hand, in the case of determining that the coefficient is the threshold value or less (NO in step S604), in step S606, the composition formula discrimination unit 120B adds the combination of the selected atomic symbol and the selected coefficient to the dopant list.

Next, in step S607, the composition formula discrimination unit 120B determines whether or not all atomic symbols included in the input composition formula have been selected. At this point, in the case of determining that not all atomic symbols have been selected (NO in step S607), the process returns to step S603.

On the other hand, in the case of determining that all atomic symbols have been selected (YES in step S607), in step S608, the composition formula discrimination unit 120B derives the formula expressing the base material by consolidating the elements included in the base material element list, or in other words, the “combinations of an atomic symbol and a new coefficient generated by rounding up the fractional part of the coefficient”.

Next, in step S609, the composition formula discrimination unit 120B determines whether or not the derived formula expressing the base material exists in the base material list. This determination is made to handle a substance that actually exists. At this point, in the case of determining that the formula expressing the base material exists in the base material list (YES in step S609), in step S610, the composition formula discrimination unit 120B outputs the formula expressing the base material and the dopant list to the descriptor computation unit 130.

On the other hand, in the case of determining that the formula expressing the base material does not exist in the base material list (NO in step S609), or in the case of determining that the sum of the coefficients of the atomic symbols included in the input composition formula is not integer (NO in step S602), in step S611, the composition formula discrimination unit 120B applies a rejection label to the input composition formula.

The material property value prediction device 100B according to Embodiment 3 and a public database were used to perform an experiment, and the result of inspecting the effect of material property prediction will be described. An overview of the specific experiment is as follows.

First, the database used as the material information was the UCSB-MRL thermoelectric database (UCSB) described in M. W. Gaultois, T. D. Sparks, C. K. H. Borg, R. Seshadri, W. D. Bonificio, and D. R. Clarke, “Data-Driven Review of Thermoelectric Materials: Performance and Resource Considerations”, Chemistry of Materials, 2013, 25, 2911-2920. This database is a public database collecting the properties of thermoelectric materials, and contains a total of 1093 materials.

Also, the predicted property values were the power factor and the electrical resistivity.

There were 456 formulas (input composition formulas) expressing a material actually used, and there were 46 formulas expressing a base material. The data used as the material information was data from which a formula expressing a base material and a formula expressing a dopant can be discriminated mechanically according to the flowchart illustrated in FIG. 23, while the formulas expressing a base material were data existing in the Inorganic Crystal Structure Database (ICSD) described in Belsky et al., from which data with attached temperature information (any of 300 K, 400 K, 700 K, and 1000 K) was chosen.

The material descriptor used in the experiment contained a descriptor indicating the temperature when measuring the properties of the material.

The material descriptor used in the experiment contained the descriptor indicating the ratio of the coefficient of an atomic symbol included in the formula expressing a dopant with respect to the sum of the coefficients of all atomic symbols included in the input composition formula described using FIG. 14.

In the case where a material i expressed by the material descriptor i used in the experiment did not contain a jth dopant, an average value was placed in the location where a descriptor for the jth dopant should be stated in the material descriptor i. Note that average value has been described in association with FIG. 16.

Also, the data was divided for each base material label such that material data containing formulas expressing the same base material did not exist in both the training data and the test data. The predicted property value was the average of the cross-validation results.

Also, power factor training method used a random forest, in which the number of trees was fixed at 500. The electrical resistivity training method used a neural network with four layers in which the number of elements in the intermediate layers was double the number of descriptors, and all of the elements were connected.

In the experiment, the root-mean-square error (RMSE) of the property values predicted according to the method in Embodiment 3 and the RMSE of the property values predicted according to the method of the related art in Furmanchuk et al. were compared.

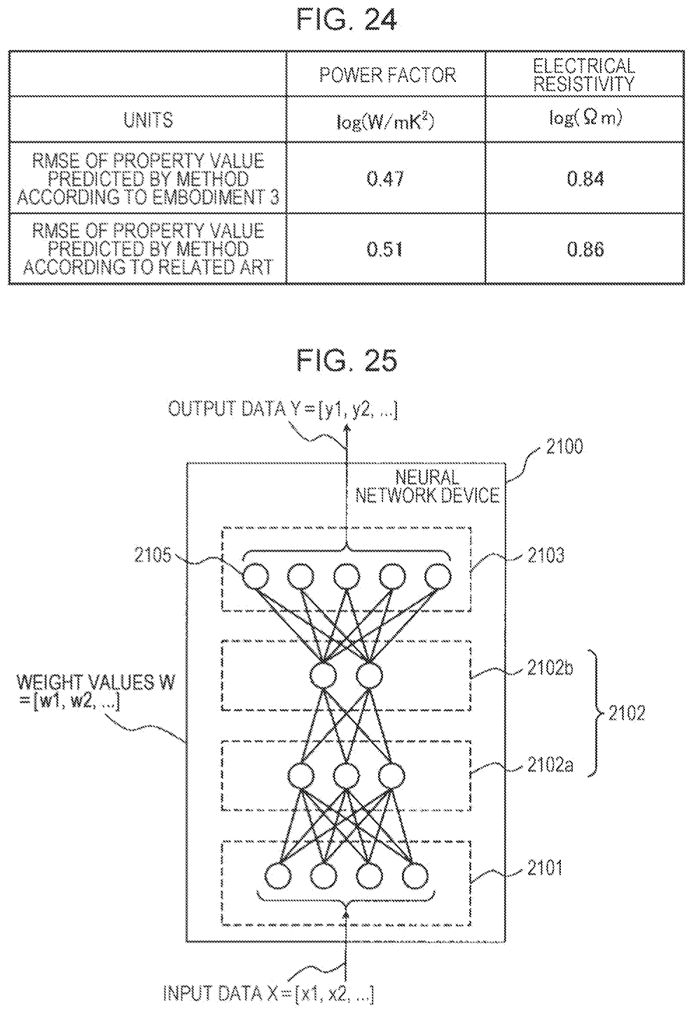

FIG. 24 is a table illustrating the results of the experiment in Embodiment 3. FIG. 24 demonstrates that the prediction accuracy is improved for both the power factor and the electrical resistivity by using the descriptors proposed in Embodiment 3.

### Embodiment 4

In the present embodiment, the predictive model of Embodiment 1 is described as a neural network device. Note that the predictive model indicated in Embodiment 2 and/or Embodiment 3 may also be the neural network device indicated in the present embodiment.

In the following, structural elements that are the same as Embodiment 1 will be denoted with the same signs, and a description thereof will be omitted. First, in preparation for describing the present embodiment, general matters related to the neural network device will be described.

FIG. 25 is a diagram explaining the concept of the neural network device in Embodiment 4. As is commonly known, a neural network device is an arithmetic device that performs arithmetic operations according to a computational model that resembles a biological neural network.

As illustrated in FIG. 25, in a neural network device 2100, units 2105 that correspond to neurons (illustrated as white circles) are arranged into an input layer 2101, a hidden layer 2102, and an output layer 2103. The hidden layer 2102 contains two hidden layers 2102a and 2102b as an example, but the hidden layer 2102 may also contain a single hidden layer or contain three or more hidden layers.

If layers near the input layer 2101 are referred to as lower layers while layers near the output layer 2103 are referred to as higher layers, the units are computational elements that perform arithmetic operations based on computational results received from units placed in a lower layer and weight values, and transmit a computational result to units placed in a higher layer.

The function of the neural network device 2100 is defined by configuration information expressing the number of layers included in the neural network device 2100 and the number of units placed in each layer, and by weight values W=[w1, w2, . . . ] expressing the weight values used in the arithmetic operations by the units.

According to the neural network device 2100, by inputting input data X=[x1, x2, . . . ] into each unit 2105 in the input layer 2101, arithmetic operations using the weight values W=[w1, w2, . . . ] are performed in the units 2105 in the hidden layer 2102 and the output layer 2103, and output data Y=[y1, y2, . . . ] is output from each unit 2105 in the output layer 2103. In FIG. 25, the output layer 2103 contains units, but the output layer may also contain a single unit, and a single piece of output data Y=y1 may be output from the single unit in the output layer.

In the following, the units 2105 placed in the input layer 2101, the hidden layer 2102, and the output layer 2103 are also referred to as the input units, the hidden units, and the output units, respectively.

In the present disclosure, the specific implementation of the neural network device 2100 is not limited. For example, the neural network device 2100 may be achieved with reconfigurable hardware or through emulation by software.

In the present disclosure, the specific method of training the neural network device 2100 is not limited. In other words, the neural network device 2100 may be trained according to a known training method other than the method described hereinafter.

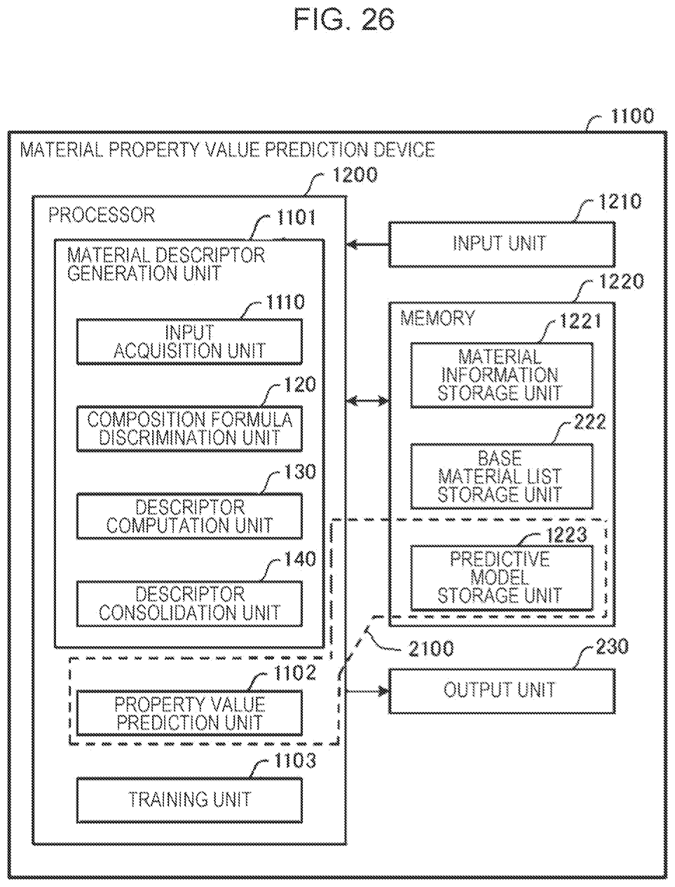

FIG. 26 is a diagram illustrating a configuration of a material property value prediction device in Embodiment 4. A material property value prediction device 1100 in Embodiment 4 includes a processor 1200, an input unit 1210, memory 1220, and an output unit 230. The processor 1200 includes a material descriptor generation unit 1101, a property value prediction unit 1102, and a training unit 1103. Additionally, the material descriptor generation unit 1101 includes an input acquisition unit 1110, a composition formula discrimination unit 120, a descriptor computation unit 130, and a descriptor consolidation unit 140. Each of the units included in the processor 1200 may also be realized as a software function exhibited by causing a microprocessor to execute a predetermined program, for example. The memory 1220 includes a material information storage unit 1221, a base material list storage unit 222, and a predictive model storage unit 1223.

Note that the predictive model includes the predictive model storage unit 1223 and the property value prediction unit 1102, and is the neural network device 2100 illustrated in FIG. 25. The material property value prediction device 1100 in Embodiment 4 is capable of switching between a training mode that trains the neural network device 2100 and a prediction mode that causes the neural network device 2100 to predict a property value of a material, according to an instruction by the user.

The operations by the material property value prediction device 1100 in the training mode and by the material property value prediction device 1100 in the prediction mode are as follows.

### <Operations by Material Property Value Prediction Device in Training Mode>

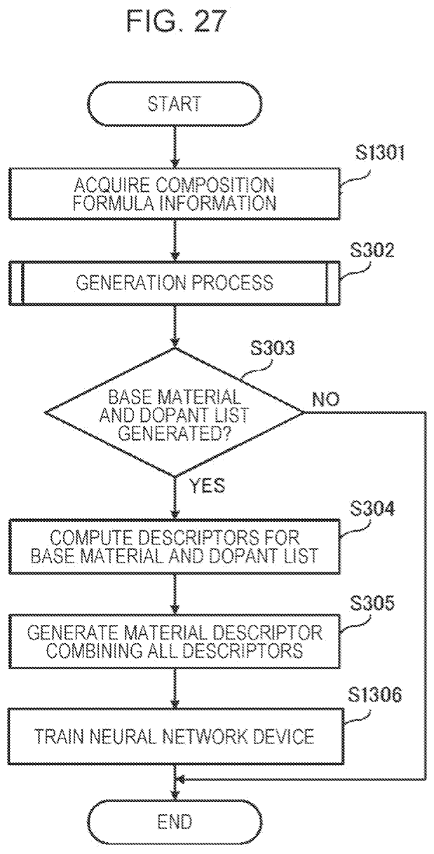

FIGS. 26 and 27 will be used to describe operations in the training mode of the material property value prediction device 1100 in Embodiment 4.

FIG. 27 is a flowchart for explaining operations in the training mode by the material property value prediction device in Embodiment 4.

The material information storage unit 1221 stores first material information in advance. The first material information includes [(composition formula of material)₁, (structure of material)₁, (environment where material is generated)₁, (property value of material)₁, . . . ] to [(composition formula of material)ₙ, (structure of material)ₙ, (environment where material is generated)ₙ, (property value of material)ₙ, . . . ]. The first material information may include one or more known parameters for each element. The known parameter(s) for each element may be an atomic volume value, a covalent radius value, or a density value.

The environment where a material is generated may be information about the temperature when generating the material and/or the temperature when measuring the properties of the material.

The property value of the material may be a value indicating the power factor of the material or a value indicating the electrical resistivity of the material.

Also, the first material information includes one or more known parameters for each element. The descriptor computation unit 130 references this information when generating a descriptor from a base material and when generating a descriptor from a dopant. The known parameter(s) for an element may be an average atomic volume value, an average covalent radius value, or an average density value.

The input unit 1210 includes a keyboard and mouse or a touch panel, for example, and receives various information input by a user.

When the input unit 1210 receives an instruction from the user to switch the material property value prediction device 1100 to the training mode, the input acquisition unit 1110 acquires (composition formula of material)₁ to (composition formula of material)ₙ included in second material information from the material information storage unit 1221 (S1301).

The predictive model storage unit 1223 includes configuration information about the neural network device 2100. The configuration information includes information indicating the number of layers included in the neural network device 2100 and the number of units placed in each layer.

The predictive model storage unit 1223 includes weight values W=[w1, w2, . . . ] used in the arithmetic operations performed by the units. Before training the neural network device 2100, the weight values W=[w1, w2, . . . ] are initial weight values Wi=[wi1, wi2, . . . ]. After training the neural network device 2100, the weight values W=[w1, w2, . . . ] are adjusted weight values Wt=[wt1, wt2, . . . ].

The property value prediction unit 1102 receives an input data X.

When the input data X is supplied to an input unit or units, the property value prediction unit 1102 performs arithmetic operations using the weight values W according to the arrangement of the units indicated by the configuration information described above.

The property value prediction unit 1102 outputs output data Y from an output unit or units. The output data Y may also be considered to be the result of the arithmetic operations performed by the output unit(s).

The training unit 1103 trains the neural network device 2100 (S1306).

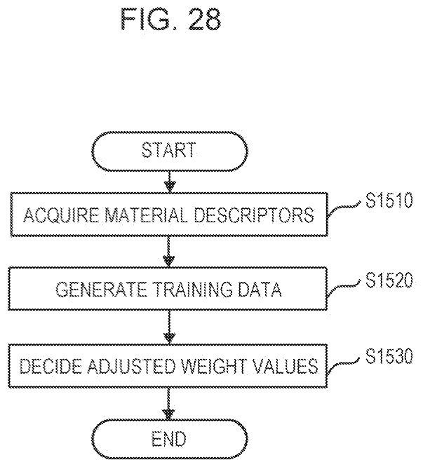

FIG. 28 is a flowchart for explaining the training process in step S1306 of FIG. 27 in Embodiment 4.

After performing a process similar to the process illustrated in steps S302 to S305 in Embodiment 1 for each of (composition formula of material)₁ to (composition formula of material)ₙ, the training unit 1103 acquires (material descriptor)₁ to (material descriptor)ₙ from the descriptor consolidation unit 140. Note that (material descriptor)₁ is generated from (composition formula of material)₁, and (material descriptor)ₙ is generated from (composition formula of material)ₙ (S1510).

The training unit 1103 references the first material information recorded in the material information storage unit 1221, and generates training data associating the material descriptor with the property value of the material. In other words, the training unit 1103 generates training data={(labeled data₁)=[(material descriptor)₁, (property value of material)₁] to (labeled dataₙ)=[(material descriptor)ₙ, (property value of material)ₙ]} (S1520).

The training unit 1103 uses the training data generated by the training unit 1103 and the initial weight values Wi=[wi1, wi2, . . . ] stored in the predictive model storage unit 1223 to decide the adjusted weight values Wt=[wt1, wt2, . . . ] by supervised learning (S1530).

With supervised learning, for example, a material descriptor included in the training data may be input into the neural network device 2100, and when output data is output by the neural network device 2100, a loss function expressing the error between the output data and the property value (that is, a label) of the material corresponding to the material descriptor may be defined, and the weight values may be updated along a gradient that decreases the value of the loss function according to a gradient descent algorithm.

Note that the operation of “inputting a material descriptor included in the training data into the neural network device 2100 and obtaining output data output by the neural network device 2100” may also be thought of as “inputting a material descriptor included in the training data into the property value prediction unit 1102 and obtaining output data output by the property value prediction unit 1102”.

Before performing the supervised learning, the weight values may also be adjusted for each layer by a form of unsupervised learning referred to as layer-wise pre-training.

With this arrangement, weight values capable of a more accurate evaluation are obtained by the subsequent supervised learning.

With unsupervised learning, for example, the input data into the neural network device 2100 and the weight values may be used to define a loss function expressing an evaluation value that does not depend on the property value of the material that acts as the label, and the weight values may be updated along a gradient that decreases the value of the loss function according to a gradient descent algorithm.

The input data to be input into the neural network device 2100 may also be subjected to data shaping processes such as normalization, thresholding, noise removal, and data size standardization. Normalization may be performed not only on the input data but also on the property value of the material that acts as the label.

Provided that the input data X=[input data into 1st unit of input layer, input data into 2nd unit of input layer, . . . ]=[x1, x2, . . . ], the input data may be input data X=[1st descriptor determined from test environment, 2nd descriptor determined from test environment, . . . , 1st descriptor determined from formula expressing base material, 2nd descriptor determined from formula expressing base material, . . . , coefficient of atomic symbol included in formula expressing 1st dopant, 1st descriptor determined from 1st dopant, 2nd descriptor determined from 1st dopant, . . . , coefficient of atomic symbol included in formula expressing nth dopant, 1st descriptor determined from nth dopant, 2nd descriptor determined from nth dopant, . . . ].

Provided that the output data Y=[output data from 1st unit of output layer]=[y1], the output data may be output data=[value indicating power factor of material expressed by input composition formula] or output data=[value indicating electrical resistivity of material expressed by input composition formula].

The 1st descriptor determined from the test environment may be information about the temperature when generating the material, and the 2nd descriptor determined from the test environment may be the temperature when measuring the properties of the material.

Instead of the coefficient of the atomic symbol included in the formula expressing the 1st dopant to the coefficient of the atomic symbol included in the formula expressing the nth dopant, the ratio of the atomic symbol included in the composition formula of the 1st dopant with respect to the sum of the coefficients of all atomic symbols included in the input composition formula to the ratio of the atomic symbol included in the composition formula of the nth dopant with respect to the sum of the coefficients of all atomic symbols included in the input composition formula may be used.

The input data may also be the above input data without the descriptors determined from the test environment, or in other words, without the 1st descriptor determined from the test environment, the 2nd descriptor determined from the test environment, and so on.

The input data may also be the above input data without the coefficient of the atomic symbol included in the formula expressing the 1st dopant to the coefficient of the atomic symbol included in the formula expressing the nth dopant.

The input data may also be the above input data without the coefficient of the atomic symbol included in the formula expressing the 1st dopant to the coefficient of the atomic symbol included in the formula expressing the nth dopant and the descriptors determined from the test environment, or in other words, the 1st descriptor determined from the test environment, the 2nd descriptor determined from the test environment, and so on.

### <Operations by Material Property Value Prediction Device in Prediction Mode>

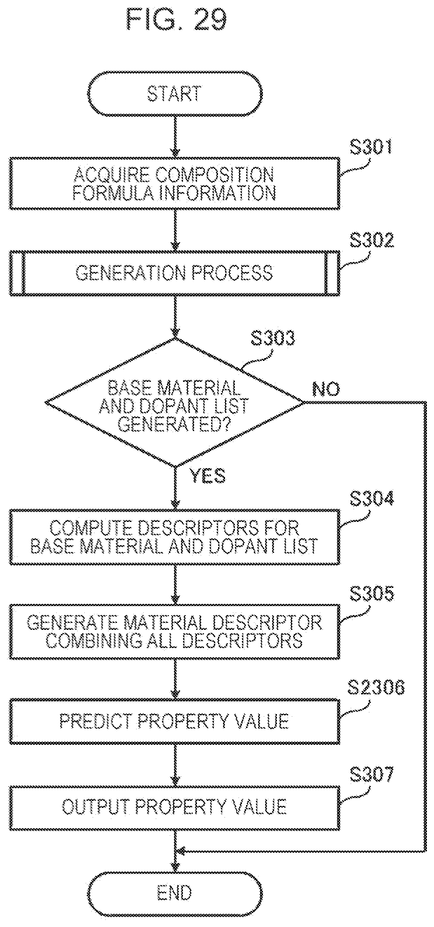

FIGS. 26 and 29 will be used to describe operations in the prediction mode of the material property value prediction device 1100 in Embodiment 4.

FIG. 29 is a flowchart for explaining operations in the prediction mode by the material property value prediction device in Embodiment 4.

After the input unit 1210 receives an instruction from the user to switch the material property value prediction device 1100 to the prediction mode, the input unit 1210 receives, from the user, the input of second material information including information about the composition formula of a material about which the user wants to predict a property value, and transmits the second material information to the input acquisition unit 1110. The input unit 1210 may also receive, from the user, the input of information indicating the structure of the material corresponding to the composition formula of the material about which the user wants to predict a property value and/or information indicating the test environment where the material corresponding to the composition formula of the material about which the user wants to predict a property value is generated, and include this information in the second material information.

The input acquisition unit 1110 receives the composition formula of the material from the input unit 1210. The composition formula of the material may also be referred to as the input composition formula.

When the neural network device 2100 receives the material descriptors generated by the descriptor consolidation unit 140 as input into the input units, the neural network device 2100 performs arithmetic operations using the adjusted weight values Wt according to the arrangement of units indicated by the configuration information stored in the predictive model storage unit 1223, and outputs a property value of the material from the output unit(s). The above operations may also be thought of as “The property value prediction unit 1102 receives the material descriptors generated by the descriptor consolidation unit 140. The property value prediction unit 1102 treats the received material descriptors as input, performs arithmetic operations using the adjusted weight values Wt according to the arrangement of units indicated by the configuration information stored in the predictive model storage unit 1223, and outputs a property value of the material.” (S2306).

With the above, the description of Embodiment 4 is concluded.

In the present disclosure, all or part of the units, devices, members, or sections, or all or part of the function blocks in the block diagram illustrated in the drawings, may also be executed by one or more electronic circuits, including a semiconductor device, a semiconductor integrated circuit (IC), or a large-scale integration (LSI) chip. An LSI chip or IC may be integrated into a single chip, or be configured by combining chips. For example, function blocks other than storage elements may be integrated into a single chip. Although referred to as an LSI chip or IC herein, such electronic circuits may also be called a system LSI chip, a very large-scale integration (VLSI) chip, or an ultra-large-scale integration (ULSI) chip, depending on the degree of integration. A field-programmable gate array (FPGA) programmed after fabrication of the LSI chip, or a reconfigurable logic device in which interconnection relationships inside the LSI chip may be reconfigured or in which circuit demarcations inside the LSI chip may be set up, may also be used for the same purpose.

Furthermore, the function or operation of all or part of a unit, device, member, or section may also be executed by software processing. In this case, the software is recorded onto a non-transitory recording medium, such as one or more ROM modules, optical discs, or hard disk drives, and when the software is executed by a processor, the function specified by the software is executed by the processor and peripheral devices. A system or device may also include one or more non-transitory recording media on which the software is recorded, a processor, and necessary hardware devices, such as an interface, for example.

In the present disclosure, the specific implementation of the predictive model is not limited. For example, the predictive model may be achieved with reconfigurable hardware or through emulation by software.

Embodiments may be obtained by making various modifications that would naturally occur to persons skilled in the art to the foregoing embodiments, and embodiments may be achieved by freely combining the structural elements and functions in the foregoing embodiments without departing from the gist of the present disclosure, but such embodiments are also included in the present disclosure.

The material descriptor generation method, material descriptor generation device, and recording medium storing a material descriptor generation program according to the present disclosure are capable of improving the performance for predicting a property value of a material, and therefore are useful as a material descriptor generation method, a material descriptor generation device, and a recording medium storing a material descriptor generation program that generate descriptors to be input into a predictive model that predicts a predetermined property value of a material.

Additionally, the predictive model construction method, predictive model construction device, and recording medium storing a predictive model construction program according to the present disclosure are capable of improving the performance for predicting a property value of a material, and therefore are useful as a predictive model construction method, a predictive model construction device, and a recording medium storing a predictive model construction program that construct a predictive model that predicts a predetermined property value of a material.

What is claimed is:

1. A material descriptor generation method comprising:
acquiring a composition formula of a material;
generating, from the composition formula, a formula expressing a base material and a dopant list including one or more formulas expressing one or more dopants used to dope the base material;
computing descriptors needed to predict a predetermined property value of the material, the descriptors corresponding to the dopant list and the formula expressing the base material; and
outputting a material descriptor consolidating the descriptors, wherein
the material descriptor is input into a predictive model that predicts the predetermined property value of the material.

2. The material descriptor generation method according to claim 1, wherein
the generating of the formula expressing the base material and the dopant list includes
acquiring a base material list including formulas expressing base materials,
computing a composition difference value between each of the formulas expressing the base materials and the composition formula,
acquiring a minimum composition difference value that is a smallest composition difference value among the computed composition difference values and a first formula expressing a first base material used to compute the minimum composition difference value, the formulas expressing the base materials including the first formula expressing the first base material,
determining whether or not the minimum composition difference value is a threshold value or less,
in a case of determining that the minimum composition difference value is greater than the threshold value, applying a rejection label to the composition formula,
in a case of determining that the minimum composition difference value is the threshold value or less, acquiring a differential composition formula expressing a formula of a difference between the first formula and the composition formula, and
generating a second formula in accordance with the differential composition formula, and
the one or more formulas expressing the one or more dopants include the second formula.

3. The material descriptor generation method according to claim 1, wherein
the generating of the formula expressing the base material and the dopant list includes
selecting an atomic symbol and a coefficient of the atomic symbol from the composition formula,
determining whether or not the coefficient is greater than a threshold value,
in a case of determining that the coefficient is the threshold value or less, adding the atomic symbol to the dopant list,
in a case of determining that the coefficient is greater than the threshold value, adding a combined formula that combines the atomic symbol with a new coefficient generated by rounding up a fractional part of the coefficient to a base material element list,
adding each atomic symbol to the dopant list or to the base material element list for all atomic symbols included in the composition formula, thereby causing the base material element list to include combined formulas, each of which is the combined formula that combines the atomic symbol with the new coefficient generated by rounding up the fractional part of the coefficient,
deriving a formula expressing a base material consolidating the combined formulas included in the base material element list, and
outputting the formula expressing the base material and the dopant list.

4. The material descriptor generation method according to claim 1, wherein
the generating of the formula expressing the base material and the dopant list includes
acquiring a base material list including formulas expressing base materials,
determining whether or not a sum of coefficients of atomic symbols in the composition formula is an integer,
in a case of determining that the sum is an integer, selecting an atomic symbol and a coefficient of the atomic symbol from the composition formula,
determining whether or not the coefficient is greater than a threshold value,
in a case of determining that the coefficient is the threshold value or less, adding the atomic symbol to the dopant list,
in a case of determining that the coefficient is greater than the threshold value, adding a combined formula that combines the atomic symbol with a new coefficient generated by rounding up a fractional part of the coefficient to a base material element list,
adding each atomic symbol to the dopant list or to the base material element list for all atomic symbols included in the composition formula, thereby causing the base material element list to include combined formulas, each of which is the combined formula that combines the atomic symbol with the new coefficient generated by rounding up the fractional part of the coefficient,
deriving a formula expressing a base material consolidating the combined formulas included in the base material element list,
determining whether or not the formula expressing the base material that is derived exists in the base material list,
in a case of determining that the formula expressing the base material exists in the base material list, outputting the formula expressing the base material and the dopant list, and
in a case of determining that the sum is not an integer, or in a case of determining that the formula expressing the base material does not exist in the base material list, applying a rejection label to the composition formula.

5. The material descriptor generation method according to claim 1, further comprising:
acquiring environment information indicating an environment where the material is generated, wherein
the computing of the descriptors includes
computing a descriptor corresponding to the environment information.

6. The material descriptor generation method according to claim 1, further comprising:
acquiring structure information indicating a structure of the material, wherein
the computing of the descriptors includes computing a descriptor corresponding to the structure information.

7. The material descriptor generation method according to claim 1, wherein
the computing of the descriptors generates a coefficient of a formula expressing a dopant included in the one or more formulas expressing the one or more dopants as a descriptor.

8. The material descriptor generation method according to claim 1, wherein
the computing of the descriptors generates, as a descriptor, a numerical value obtained by dividing each of one or more coefficients of the one or more formulas expressing the one or more dopants included in the dopant list by a sum of all coefficients included in the composition formula.

9. The material descriptor generation method according to claim 1, wherein
in a case where a second coefficient is decreased due to increasing a first coefficient, the computing of the descriptors generates a coefficient indicating an amount of the decrease as a descriptor, and
the one or more formulas expressing the one or more dopants includes a first atomic symbol having the first coefficient and a second atomic symbol having the second coefficient.

10. A material descriptor generation device comprising:
an acquirer that acquires a composition formula of a material;
a discriminator that discriminates, from the composition formula, a formula expressing a base material and a dopant list including one or more formulas expressing one or more dopants used to dope the base material;
a calculator that computes descriptors needed to predict a predetermined property value of the material, the descriptors corresponding to the dopant list and the formula expressing the base material; and
an outputter that outputs a material descriptor consolidating the descriptors, wherein
the material descriptor is input into a predictive model that predicts the predetermined property value of the material.

11. A non-transitory computer-readable recording medium storing a material descriptor generation program that causes a computer to execute a process comprising:
acquiring a composition formula of a material;
generating, from the composition formula, a formula expressing a base material and a dopant list including one or more formulas expressing one or more dopants used to dope the base material;
computing descriptors needed to predict a predetermined property value of the material, the descriptors corresponding to the dopant list and the formula expressing the base material; and
outputting a material descriptor consolidating the descriptors, wherein
the material descriptor is input into a predictive model that predicts the predetermined property value of the material.

12. A predictive model construction method in a predictive model construction device that constructs a predictive model predicting a predetermined property value of a material, comprising:
generating a descriptor indicating a predetermined feature of the material; and
training the predictive model by using the descriptor as an input value.

13. The predictive model construction method according to claim 12, wherein
the generating of the descriptor includes
acquiring a composition formula of the material,
generating, from the composition formula, a formula expressing a base material and a dopant list including one or more formulas expressing one or more dopants used to dope the base material,
computing descriptors needed to predict the predetermined property value, the descriptors corresponding to the dopant list and the formula expressing the base material, and
outputting a material descriptor consolidating the descriptors.

14. A predictive model construction device that constructs a predictive model predicting a predetermined property value of a predetermined material, comprising:
a generator that generates a descriptor indicating a feature of the predetermined material; and
a trainer that trains the predictive model by using the descriptor as an input value.

15. A non-transitory computer-readable recording medium storing a predictive model construction program that causes a computer to execute a process of constructing a predictive model predicting a predetermined property value of a predetermined material, the process comprising:
generating a descriptor indicating a feature of the predetermined material; and
training the predictive model by using the descriptor as an input value.
